(19) United States
(12) Patent Application Publication
(10) Pub. No.: US 2005/0137194 A1
(43) Pub. Date: Jun. 23, 2005

(54) Combination of selected opioids with other active compounds for treatment of urinary incontinence

(75) Inventor: Thomas Cristoph, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/998,164

(22) Filed: Nov. 29, 2004

Related U.S. Application Data
(63) Continuation of application No. PCT/EP03/05529, filed on May 27, 2003.

(30) Foreign Application Priority Data
May 29, 2002 (DE) 102 24 107.4

(51) Int. Cl. A61K 31/5377; A61K 31/485

(52) U.S. Cl. 514/235.2; 514/282

(57) ABSTRACT

The invention relates to the combination of compounds of group A, especially opioids, with compounds of group B for for the treatment of urinary urgency or urinary incontinence. The invention also relates to corresponding pharmaceutical formulations and to methods for treating urinary urgency or urinary incontinence with a compound of group A and a compound of group B.

### CROSS REFERENCE TO RELATED APPLICATIONS

This application is a continuation of International Patent Application No. PCT/EP03/05529, filed May 27, 2003, designating the United States of America, and published in German as WO 03/099268 A1, the entire disclosure of which is incorporated herein by reference. Priority is claimed based on Federal Republic of Germany Patent Application No. 102 24 107.4, filed May 29, 2002.

### FIELD OF THE INVENTION

The invention relates to the use of a combination of compounds of group A, in particular opioids, and compounds of group B, for the preparation of a medicament for treatment of an increased urge to urinate and urinary incontinence, and corresponding medicaments and methods for treatment of an increased urge to urinate and urinary incontinence.

### BACKGROUND OF THE INVENTION

Urinary incontinence is the involuntary discharge of urine. This occurs in an uncontrolled manner when the pressure within the urinary bladder exceeds that needed to close the ureter. Causes can be on the one hand an increased internal bladder pressure (e.g. due to detrusor instability) with the consequence of urgency incontinence and on the other hand a reduced sphincter pressure (e.g. following giving birth or surgical interventions) with the consequence of stress incontinence. The detrusor is a coarsely bundled multilayered bladder wall musculature, contraction of which leads to voiding of urine, and the sphincter is the muscle which closes the urethra. Mixed forms of these types of incontinence as well as so-called overflow incontinence (e.g. in cases of benign prostate hyperplasia) or reflex incontinence (e.g. following damage to the spinal marrow) occur. Further details in this context are to be found in Chutka, D. S. and Takahashi, P. Y., 1998, Drugs 560: 587-595.

The urge to urinate is the state of increased bladder muscle tension as the bladder capacity is approached (or when this is exceeded), the aim of which is voiding of urine (micturition). This tensioning acts as a stimulus to micturition. An increased urge to urinate is understood in this context in particular as meaning the occurrence of a premature or more frequent and sometimes even painful urge to urinate. This leads as a consequence to significantly more frequent micturition. Causes can be, inter alia, inflammation of the urinary bladder and neurogenic bladder disorders and also bladder tuberculosis. However, all the causes have not yet been clarified.

An increased urge to urinate and also urinary incontinence are found to be extremely unpleasant, and there is a clear need to achieve an improvement in persons affected by these indications which is as long-term as possible.

An increased urge to urinate and in particular urinary incontinence are conventionally treated with substances which are involved in the reflexes of the lower urinary tract (Wein, A. J., 1998, Urology 51 (Suppl. 21): 43-47). These are usually medicaments which have an inhibiting action on the detrusor muscle, which is responsible for the internal bladder pressure. These medicaments are e.g. parasympatholytics, such as oxybutynin, propiverine or tolterodine, tricyclic antidepressants, such as imipramine, or muscle relaxants, such as flavoxate. Other medicaments which in particular increase the resistance of the urethra or of the neck of the bladder show affinities for α-adrenoreceptors, such as ephedrine, to β-adrenoreceptors, such as clenbuterol, or are hormones, such as oestradiol.

An accurate insight into the therapeutics and treatment methods used, in particular in respect of the antimuscarinics and other peripherally acting substances, is given in this context by the review article by K. E. Andersson et al. “The pharmacological treatment of urinary incontinence”, BJU International (1999), 84, 923-947.

Certain diarylmethylpiperazines and -piperidines are also described for this indication in WO 93/15062. A positive effect on bladder function has also been demonstrated for tramadol in a rat model of rhythmic bladder contractions (Nippon-Shinyaku, WO 98/46216). Furthermore, there are investigations in the literature for characterization of the opioid side effect of urine retention, from which emerge some indications of influencing of bladder functions by weak opioids, such as diphenoxylate (Fowler et al., 1987, J. Urol 138: 735-738) and meperidine (Doyle and Briscoe, 1976, Br J Urol 48: 329-335), by mixed opioid agonists/antagonists, such as buprenorphine (Malinovsky et al., 1998 Anesth Analg 87: 456-461; Drenger and Magora, 1989 Anesth Analg 69: 348-353), pentazocine (Shimizu et al. (2000) Br. J. Pharmacol. 131 (3): 610-616 and nalbuphine (Malinovsky et al., 1998, loc. cit.), and by potent opioids, such as morphine (Malinovsky et al., 1998 loc. cit.; Kontani and Kawabata, (1988); Jpn J. Pharmacol. Sep.; 48(1):31) and fentanyl (Malinovsky et al., 1998 loc. cit.). However, these investigations were usually carried out in analgesically active concentrations.

With the indications in question here, it should be remembered that it is a matter in general of very long-term uses of medicaments and, in contrast to many situations in which analgesics are employed, those affected are faced with a situation which is very unpleasant but not unendurable. It should therefore be ensured here—even more so than with analgesics—that side effects are avoided if the person affected does not want to replace one evil by the other. Furthermore, analgesic actions are also largely undesirable during long-term urinary incontinence treatment.

### SUMMARY OF THE INVENTION

One object of the present invention was therefore to discover substances or substance combinations which are helpful for treatment of an increased urge to urinate and urinary incontinence, and in the active doses preferably at the same time show a lower degree of side effects and/or analgesic actions than known from the prior art. Preferably, the combinations show a synergistic effect for treatment of urinary incontinence.

Surprisingly, it has now been found that a combination of compounds from group A, which comprises opioids and other centrally acting substances which interact with opioid receptors, the effects of which can be antagonized by naloxone, or in particular substances which act via an opiate receptor, in particular the μ receptor, and compounds of group B, which comprises muscarine antagonists and other predominantly peripherally acting substances which are known to be active in urinary incontinence, have an outstanding action on bladder function. Furthermore, these combinations are highly and significantly, unexpectedly, active at very low doses so that it is possible to employ the combined active compounds in a low dose. As a result, it is to be expected that side effects which otherwise occur at the higher necessary dosages will decrease significantly, while the therapeutic action is fully retained by this combination of peripheral antimuscarinic effect acting predominantly directly on the bladder or bladder musculature and central opioid effect or μ receptor effect.

The invention accordingly provides the use of an active compound combination, or pharmaceutical formulation of at least one of the compounds A and at least one of the compounds B, where compound A is chosen from:
Group a) comprising:
tramadol, O-demethyltramadol or O-demethyl-N-monodemethyl-tramadol as the free base or acid and/or in the form of physiologically acceptable salts, in particular in the form of their physiologically acceptable acidic and basic salts or salts with cations and bases or with anions and acids; in the form of the enantiomers, diastereomers, in particular mixtures of their enantiomers or diastereomers, or an individual enantiomer or diastereomer;
Group b) comprising:
   codeine
   dextropropoxyphene
   dihydrocodeine
   diphenoxylate
   ethylmorphine
   meptazinol
   nalbuphine
   pethidine (meperidine)
   tilidine
   tramadol
   viminol
   butorphanol
   dextromoramide
   dezocine
   diacetylmorphine (heroin)
   hydrocodone
   hydromorphone
   ketobemidone
   levomethadone
   levomethadyl acetate (1-α-acetylmethadol (LAAM))
   levorphanol
   morphine
   nalorphine
   oxycodone
   pentazocine
   piritramide
   alfentanil
   buprenorphine
   etorphine
   fentanyl
   remifentanil
   sufentanil
as the free base or acid and/or in the form of physiologically acceptable salts, in particular in the form of their physiologically acceptable acidic and basic salts or salts with cations and bases or with anions and acids, optionally in the form of the enantiomers, diastereomers, in particular mixtures of their enantiomers or diastereomers, or an individual enantiomer or diastereomer;
Group c) comprising:
1-phenyl-3-dimethylamino-propane compounds according to the general formula I 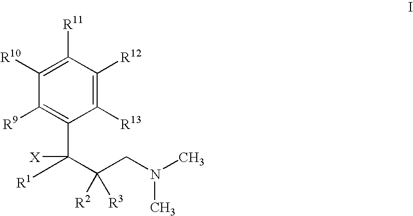
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted,
R¹ is chosen from C₁₋₄-alkyl, branched or unbranched, saturated or unsaturated, unsubstituted or mono- or polysubstituted,
R² and R³ in each case independently of one another are chosen from H or C₁₋₄-alkyl, branched or unbranched, saturated or unsaturated, unsubstituted or mono- or polysubstituted, or
R² and R³ together form a saturated C₄₋₇-cycloalkyl radical, unsubstituted or mono- or polysubstituted,
R⁹ to R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄—(C₁₋₃-alkyl), CO(C₁₋₅-alkyl), CO_CHR¹⁷—NHR¹⁸, CO—C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring,
as the free base or acid and/or in the form of physiologically acceptable salts, in particular in the form of their physiologically acceptable acidic and basic salts or salts with cations and bases or with anions and acids; in the form of the enantiomers, diastereomers, in particular mixtures of their enantiomers or diastereomers, or an individual enantiomer or diastereomer;
Group d) comprising:
substituted 6-dimethylaminomethyl-1-phenylcyclohexane compounds according to the general formula II 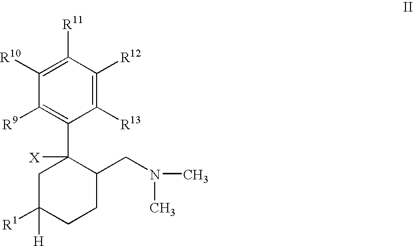
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted,
R¹ is chosen from C₁₋₄-alkyl, benzyl, CF₃, OH, OCH₂—C₆H₅, O—C₁₋₄-alkyl, Cl or F and
R⁹ to R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄—(C₁₋₃-alkyl), CO(C₁₋₅-alkyl), CO—CHR¹⁷—NHR¹⁸, C₀-C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring,
as the free base or acid and/or in the form of physiologically acceptable salts, in particular in the form of their physiologically acceptable acidic and basic salts or salts with cations and bases or with anions and acids; in the form of the enantiomers, diastereomers, in particular mixtures of their enantiomers or diastereomers, or an individual enantiomer or diastereomer; and/or
Group e) comprising:
6-dimethylaminomethyl-1-phenyl-cyclohexane compounds according to the general formula III 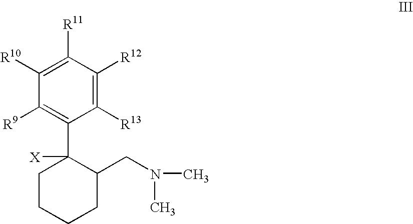
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted, and
R⁹ to R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄—(C₁₋₃-alkyl), CO(C₁₋₅-alkyl), CO—CHR¹⁷—NHR¹⁸, CO—C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring,
with the proviso that if R⁹, R¹¹ and R¹³ correspond to H and one of R¹⁰ or R¹² corresponds to H and the other corresponds to OCH₃, X may not be OH,
as the free base or acid and/or in the form of physiologically acceptable salts, in particular in the form of their physiologically acceptable acidic and basic salts or salts with cations and bases or with anions and acids; in the form of the enantiomers, diastereomers, in particular mixtures of their enantiomers or diastereomers, or an individual enantiomer or diastereomer;
and with at least one of the compounds B chosen from
the antimuscarinics: atropine, oxybutynin, propiverine, propantheline, emepronium, trospium, tolterodine, darifenacin and α,α-diphenylacetic acid 4-(N-methylpiperidyl) ester, as well as duloxetine, imipramine and desmopressin, and
venlafaxine, fesoterodine, solifenacin (YM905), resiniferatoxin, cizolirtine, nitro-flurbiprofen, HCT1026, talnetant, TAK-637, SL 251039, R 450, Rec 15/3079, (−)-DDMS, NS-8 and/or DRP-001
as the free base or acid and/or in the form of physiologically acceptable salts, in particular in the form of their physiologically acceptable acidic and basic salts or salts with cations and bases or with anions and acids, optionally in the form of the enantiomers, diastereomers, in particular mixtures of their enantiomers or diastereomers, or an individual enantiomer or diastereomer;
for the preparation of a medicament for treatment of an increased urge to urinate and urinary incontinence.

Surprisingly, it had been found that the combination of the substances mentioned significantly positively influence certain physiological parameters which are of importance in cases of an increased urge to urinate and urinary incontinence. Each of these individual changes can mean a significant alleviation of the symptomatic picture of patients affected.

The compounds of group B predominantly have a peripheral action on urinary incontinence. In this context, venlafaxine is a selective noradrenalin reuptake inhibitor having an activity in stress incontinence (Bae J. H. et al., BJU International 2001, 88, 771, 775). Fesoterodine is an mACh antagonist developed by Schwarz Pharma. Solifenacin (YM905) is an mACh antagonist developed by Yamanouchi. Resiniferatoxin is a VR1 agonist developed by Afferon, Mundipharma and ICOS (although in particular for local use). Cizolirtine is a compound described in European Patent EP 289 380 B1 (2-[phenyl(1-methyl-1H-pyrazol-5-yl)methoxy]-N,N-dimethylethanamine, which can also be called 5-[alpha-(2-dimethylaminoethoxy)benzyl]-1-methyl-1H-pyrazole or 5-[N,N-(dimethylaminoethoxy)phenyl]methyl-1-methyl-1H-pyrazole) with a hitherto unknown action mechanism, which is being investigated clinically in urinary incontinence by Esteve (ES). Nitro-flurbiprofen and HCT-1026 are two substances which act on NO+COX and have been developed by NicOx. Talnetant is an NK antagonist developed by Glaxo Smith Kline. TAK-637 is an NK antagonist developed by Takeda. SL 251039 is an a₁AR agonist developed by Sanofi. R450 is an a₁AR agonist developed by Roche. Rec 15/3079 is a 5HT_{1A} antagonist developed by Recordati. (−)-DDMS is a substance developed by Sepracor which acts on NA+D. NS-8 is a substance developed by Nippon Shinyaku which acts on PCA. DRP-001 is a substance of unknown action mechanism developed by Sosei for urgency incontinence.

In the context of this invention, alkyl and cycloalkyl radicals are understood as meaning saturated and unsaturated (but not aromatic), branched, unbranched and cyclic hydrocarbon radicals, which can be unsubstituted or mono- or polysubstituted. In this context, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4- or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C₁₋₈-alkyl. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. In respect of cycloalkyl, the term also includes saturated cycloalkyls in which one or 2 carbon atoms are replaced by a heteroatom S, N or O. However, the term cycloalkyl also includes in particular mono- or poly-, preferably monounsaturated cycloalkyls without a heteroatom in the ring as long as the cycloalkyl is not an aromatic system. Preferably, the alkyl and cycloalkyl radicals are methyl, ethyl, vinyl (ethenyl), propyl, allyl (2-propenyl), 1-propynyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentyl, cyclopentylmethyl, cyclohexyl, cycloheptyl, cyclooctyl and also adamantyl, CHF₂, CF₃ or CH₂OH, as well as pyrazolinone, oxopyrazolinone, [1,4]dioxane or dioxolane.

In this context, in connection with alkyl and cycloalkyl—as long as this is not expressly defined otherwise—the term substituted in the context of this invention is understood as meaning substitution of at least one (optionally also several) hydrogen radical(s) by F, Cl, Br, I, NH₂, SH or OH, where “polysubstituted” or “substituted” in the case of multiple substitution is to be understood as meaning that the substitution is both on different and on the same atoms several times with the same or different substituents, for example three times on the same C atom as in the case of CF₃ or in various places as in the case of —CH(OH)—CH═CH—CHCl₂. Particularly preferred substituents here are F, Cl and OH. In respect of cycloalkyl, the hydrogen radical can also be replaced by OC₁₋₃-alkyl or C₁₋₃-alkyl (in each case mono- or polysubstituted or unsubstituted), in particular methyl, ethyl, n-propyl, i-propyl, CF₃, methoxy or ethoxy.

Pharmaceutical formulation means that that ingredients are prepared for coadministration.

The term (CH₂)₃₋₆ is to be understood as meaning —CH₂—CH₂—CH₂—, —CH₂—CH₂—CH₂—CH₂—, —CH₂—CH₂—CH₂—CH₂—CH₂— and —CH₂—CH₂—CH₂—CH₂—CH₂—CH₂—, (CH₂)₁₄ is to be understood as meaning —CH₂—, —CH₂—CH₂—, —CH₂—CH₂—CH₂— and —CH₂—CH₂—CH₂—CH₂—, (CH₂)₄₋₅ is to be understood as meaning —CH₂—CH₂—CH₂—CH₂— and —CH₂—CH₂—CH₂—CH₂—CH₂— etc.

An aryl radical is understood as meaning ring systems having at least one aromatic ring but without heteroatoms in even only one of the rings. Examples are phenyl, naphthyl, fluoranthenyl, fluorenyl, tetralinyl or indanyl, in particular 9H-fluorenyl or anthracenyl radicals, which can be unsubstituted or mono- or polysubstituted.

A heteroaryl radical is understood as meaning heterocyclic ring systems having at least one unsaturated ring, which contain one or more heteroatoms from the group consisting of nitrogen, oxygen and/or sulfur and can also be mono- or polysubstituted. Examples which may be mentioned from the group of heteroaryls are furan, benzofuran, thiophene, benzothiophene, pyrrole, pyridine, pyrimidine, pyrazine, quinoline, isoquinoline, phthalazine, benzo-1,2,5-thiadiazole, benzothiazole, indole, benzotriazole, benzodioxolane, benzodioxane, carbazole, indole and quinazoline.

In this context, in connection with aryl and heteroaryl, substituted is understood as meaning substitution of the aryl or heteroaryl by R²³, OR²³, a halogen, preferably F and/or Cl, a CF₃, a CN, an NO₂, an NR²⁴R²⁵, a C₁₋₆-alkyl (saturated), a C₁₋₆-alkoxy, a C₃₋₈-cycloalkoxy a C₃₋₈-cycloalkyl or a C₂₋₆-alkylene.

In this context, the radical R²³ represents H, a C₁₋₁₀-alkyl, preferably a C₁₋₆-alkyl, or an aryl or heteroaryl radical or an aryl or heteroaryl radical bonded via a C₁₋₃-alkylene group, where these aryl and heteroaryl radicals may not themselves be substituted by aryl or heteroaryl radicals
the radicals R²⁴ and R²⁵ are identical or different and denote H, a C₁₋₁₀-alkyl, preferably a C₁₋₆-alkyl, or an aryl or a heteroaryl radical or an aryl or heteroaryl radical bonded via a C₁₋₃-alkylene group, where these aryl and heteroaryl radicals may not themselves be substituted by aryl or heteroaryl radicals
or the radicals R²⁴ and R²⁵ together denote CH₂CH₂OCH₂CH₂, CH₂CH₂NR²⁶ CH₂CH₂ or (CH₂)₃₋₆, and
the radical R²⁶ represents H, a C₁₋₁₀-alkyl, preferably a C₁₋₆-alkyl, or an aryl or heteroaryl radical, or represents an aryl or heteroaryl radical bonded via a C₁₋₃-alkylene group, where these aryl and heteroaryl radicals may not themselves be substituted by aryl or heteroaryl radicals.

The term salt is to be understood as meaning any form of the active compound according to the invention in which this assumes an ionic form or is charged and is coupled with a counter-ion (a cation or anion) or is in solution. This is also to be understood as meaning complexes of the active compound with other molecules and ions, in particular complexes which are complexed via ionic interactions.

The term of the physiologically acceptable salt with cations or bases in the context of this invention is understood as meaning salts of at least one of the compounds according to the invention—usually a (deprotonated) acid—as the anion with at least one, preferably inorganic cation, which are physiologically acceptable—in particular when used in humans and/or mammals. Particularly preferred salts are the salts of the alkali metals and alkaline earth metals, but also with NH₄⁺, but in particular (mono)- or (di)-sodium, (mono)- or (di)-potassium, magnesium or calcium salts.

The term of the physiologically acceptable salt with anions or acids is understood in the context of this invention as meaning salts of at least one of the compounds according to the invention—usually protonated, for example on the nitrogen—as the cation with at least one anion which are physiologically acceptable—in particular when used in humans and mammals. In particular, in the context of this invention this is understood as meaning the salt formed with a physiologically acceptable acid, namely salts of the particular active compound with inorganic or organic acids which are physiologically acceptable—in particular when used in humans and/or mammals. Examples of physiologically acceptable salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1,2-dihydroλ⁶-benzo[d]isothiazol-3-one (saccharic acid), monomethylsebacic acid, 5-oxo-proline, hexane-1-sulfonic acid, nicotinic acid, 2-, 3- or 4-aminobenzoic acid, 2,4,6-trimethylbenzoic acid, α-liponic acid, acetylglycine, acetylsalicylic acid, hippuric acid and/or aspartic acid. The hydrochloride salt is particularly preferred.

Suitable salts in the context of this invention and in each use described and each of the medicaments described are salts of the particular active compound with inorganic or organic acids and/or a sugar substitute, such as saccharin, cyclamate or acesulfame. However, the hydrochloride is particularly preferred.

Compounds of group c) and their preparation are known from DE 44 26 245 A1 and U.S. Pat. No. 6,248,737. Compounds of group d) and e) and their preparation are known from DE 195 25 137 A1 and U.S. Pat. No. 5,733,936 and US RE37355E.

In a preferred embodiment, for the use according to the invention the compound A in group a) is chosen from:
tramadol, (+)-tramadol, (+)—O-demethyltramadol or (+)—O-demethyl-N-mono-demethyl-tramadol, preferably tramadol or (+)-tramadol, in particular (+)-tramadol.

In a preferred embodiment, for the use according to the invention the compound A in group b) is chosen from:
codeine
dextropropoxyphene
dihydrocodeine
diphenoxylate
ethylmorphine
meptazinol
nalbuphine
pethidine (meperidine)
tilidine
viminol
butorphanol
dezocine
nalorphine
pentazocine
buprenorphine
   preferably
codeine
dextropropoxyphene
dihydrocodeine
meptazinol
nalbuphine
tilidine
buprenorphine

In a preferred embodiment, for the use according to the invention the compound A in group c) is chosen from compounds according to formula I for which:
X is chosen from
OH, F, Cl, OC(O)CH₃ or H, preferably OH, F, OC(O)CH₃ or H, and/or
R¹ is chosen from
C₁₋₄-alkyl, saturated and unsubstituted, branched or unbranched; preferably CH₃, C₂H₅, C₄H₉ or t-butyl, in particular CH₃ or C₂H₅, and/or
R² and R³ independently of one another are chosen from
H or C₁₋₄-alkyl, saturated and unsubstituted, branched or unbranched; preferably H, CH₃, C₂H₅, i-propyl or t-butyl, in particular H or CH₃, preferably R³═H, or
R² and R³ together form a C₅₋₆-cycloalkyl radical, saturated or unsaturated, unsubstituted or mono- or polysubstituted, preferably saturated and unsubstituted, in particular cyclohexyl, and/or
R⁹ to R¹³, where 3 or 4 of the radicals R⁹ to R¹³ must correspond to H, independently of one another are chosen from
H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
preferably H, Cl, F, OH, CF₂H, CF₃, OCH₃ or SCH₃
or R¹² and R¹¹ form a 3,4-OCH═CH ring
in particular
if R⁹, R¹¹ and R¹³ correspond to H, one of R¹⁰ or R¹² also corresponds to H, while the other is chosen from:
Cl, F, OH, CF₂H, CF₃, OR¹⁴ or SR¹⁴, preferably OH, CF₂H, OCH₃ or SCH₃ or
if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F, preferably Cl, or
if R⁹, R¹⁰, R¹² and R¹³ correspond to H, R¹¹ is chosen from CF₃, CF₂H, Cl or F, preferably F, or
if R¹⁰, R¹¹ and R¹² correspond to H, one of R⁹ or R¹³ also corresponds to H, while the other is chosen from OH, OC₂H₅ or OC₃H₇.

In this context, for compounds of group c) it is particularly preferable if compounds of the formula I where R³═H are present in the form of the diastereomers having the relative configuration Ia 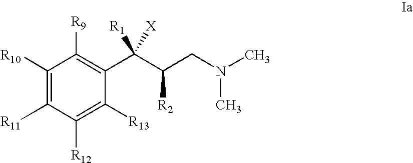
in particular are used in mixtures having a higher content of this diastereomer compared with the other diastereomer or as the pure diastereomer and/or
if the compounds of the formula I are used in the form of the (+)-enantiomer, in particular in mixtures having a higher content of the (+)-enantiomer compared with the (−)-enantiomer of a racemic compound or as the pure (+)-enantiomer.

In this context, it is particularly preferable if compound A chosen from the following group is used:
(2RS,3RS)-1-dimethylamino-3-(3-methoxy-phenyl)-2-methyl-pentan-3-ol,
(+)-(2R,3R)-1-dimethylamino-3-(3-methoxy-phenyl)-2-methyl-pentan-3-ol,
(2RS,3RS)-3-(3,4-dichlorophenyl)-1-dimethylamino-2-methyl-pentan-3-ol,
(2RS,3RS)-3-(3-difluoromethyl-phenyl)-1-dimethylamino-2-methyl-pentan-3-ol,
(2RS,3RS)-1-dimethylamino-2-methyl-3-(3-methylsulfanyl-phenyl)-pentan-3-ol,
(3RS)-1-dimethylamino-3-(3-methoxy-phenyl)-4,4-dimethyl-pentan-3-ol,
(2RS,3RS)-3-(3-dimethylamino-1-ethyl-1-hydroxy-2-methyl-propyl)-phenol,
(1RS,2RS)-3-(3-dimethylamino-1-hydroxy-1,2-dimethyl-propyl)-phenol,
(+)-(1R,2R)-3-(3-dimethylamino-1-hydroxy-1,2-dimethyl-propyl)-phenol,
(+)-(1R,2R)-3-(3-dimethylamino-1-hydroxy-1,2-dimethyl-propyl)-phenol,
(−)-(1R,2R)-3-(3-dimethylamino-1-ethyl-2-methyl-propyl)-phenol,
(+)-(1R,2R)-acetic acid 3-dimethylamino-1-ethyl-1-(3-methoxy-phenyl)-2-methyl-propyl ester,
(1RS)-1-(1-dimethylaminomethyl-cyclohexyl)-1-(3-methoxy-phenyl)-propan-1-ol,
(2RS,3RS)-3-(4-chlorophenyl)-1-dimethylamino-2-methyl-pentan-3-ol,
(+)-(2R,3R)-3-(3-dimethylamino-1-ethyl-1-hydroxy-2-methyl-propyl-phenol,
(2RS,3RS)-4-dimethylamino-2-(3-methoxy-phenyl)-3-methyl-butan-2-ol and
(+)-(2R,3R)-4-dimethylamino-2-(3-methoxy-phenyl)-3-methyl-butan-2-ol,
   preferably as the hydrochloride.

In a preferred embodiment, for the use according to the invention the compound A in group d) is chosen from compounds according to formula II for which:
X is chosen from
OH, F, Cl, OC(O)CH₃ or H, preferably OH, F or H, in particular OH, and/or
R¹ is chosen from
C₁₋₄-alkyl, CF₃, OH, O—C₁₋₄-alkyl, Cl or F, preferably OH, CF₃ or CH₃, and/or
R⁹ to R¹³, where 3 or 4 of the radicals R⁹ to R¹³ must correspond to H, independently of one another are chosen from
H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
preferably H, Cl, F, OH, CF₂H, CF₃, OCH₃ or SCH₃
or R¹² and R¹¹ form a 3,4-OCH═CH ring
in particular
if R⁹, R¹¹ and R¹³ correspond to H, one of R¹⁰ or R¹² also corresponds to H, while the other is chosen from:
Cl, F, OH, CF₂H, CF₃, OR¹⁴ or SR¹⁴, preferably OH, CF₂H, OR¹⁴ or SCH₃, in particular OH or OC₁₋₃-alkyl, preferably OH or OCH₃, or
if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F, preferably Cl, or
if R⁹, R¹⁰, R¹² and R¹³ correspond to H, R¹¹ is chosen from CF₃, CF₂H, Cl or F, preferably F, or
if R¹⁰, R¹¹ and R¹² correspond to H, one of R⁹ or R¹³ also corresponds to H, while the other is chosen from OH, OC₂H₅ or OC₃H₇.
very particularly preferably
if R⁹, R¹¹ and R¹³ correspond to H, one of R¹⁰ or R¹² also corresponds to H, while the other is chosen from:
Cl, F, OH, SH, CF₂H, CF₃, OR¹⁴ or SR¹⁴, preferably OH or OR¹⁴, in particular OH or OC₁₋₃-alkyl, preferably OH or OCH₃.

In this context, for compounds of group d) it is particularly preferable if compounds of the formula II are present in the form of the diastereomers having the relative configuration IIa 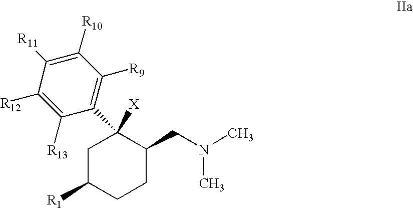
in particular, where they are used in mixtures having a higher content of this diastereomer compared with the other diastereomer or as the pure diastereomer and/or
   if the compounds of the formula II are used in the form of the (+)-enantiomer, in particular in mixtures having a higher content of the (+)-enantiomer compared with the (−)-enantiomer of a racemic compound or as the pure (+)-enantiomer.

In this context, it is particularly preferable if compound A chosen from the following group is used:
(1RS,3RS,6RS)-6-dimethylaminomethyl-1-(3-methoxy-phenyl)-cyclohexane-1,3-diol,
(+)-(1R,3R,6R)-6-dimethylaminomethyl-1-(3-methoxy-phenyl)-cyclohexane-1,3-diol,
(1RS,3RS,6RS)-6-dimethylaminomethyl-1-(3-hydroxy-phenyl)-cyclohexane-1,3-diol,
(1RS,3 SR,6RS)-6-dimethylaminomethyl-1-(3-methoxy-phenyl)-cyclohexane-1,3-diol,
(+)-(1R,2R,5 S)-3-(2-dimethylaminomethyl-1-hydroxy-5-methyl-cyclohexyl)-phenol or
(1RS,2RS,5RS)-3-(2-dimethylaminomethyl-1-hydroxy-5-trifluoromethyl-cyclohexyl)-phenol,
   preferably as the hydrochloride.

In a preferred embodiment, for the use according to the invention the compound A in group e) is chosen from compounds according to formula III for which:
X is chosen from
OH, F, Cl, OC(O)CH₃ or H, preferably OH, F or H, in particular F or H, and/or
R⁹ to R¹³, where 3 or 4 of the radicals R⁹ to R¹³ must correspond to H, independently of one another are chosen from
H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
preferably H, Cl, F, OH, CF₂H, CF₃, OCH₃ or SCH₃
or R¹² and R¹¹ form a 3,4-OCH═CH ring
in particular characterized in that
if R⁹, R¹¹ and R¹³ correspond to H, one of R¹⁰ or R¹² also corresponds to H, while the other is chosen from:
Cl, F, OH, CF₂H, CF₃, OR¹⁴ or SR¹⁴, preferably OH, CF₂H, OR¹⁴ or SCH₃, in particular OH or OC₁₋₃-alkyl, preferably OH or OCH₃, or
if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F, preferably Cl, or
if R⁹, R¹⁰, R¹² and R¹³ correspond to H, R¹¹ is chosen from CF₃, CF₂H, Cl or F, preferably F, or
if R¹⁰, R¹¹ and R¹² correspond to H, one of R⁹ or R¹³ also corresponds to H, while the other is chosen from OH, OC₂H₅ or OC₃H₇.
very particularly preferably
if R⁹, R¹¹ and R¹³ correspond to H, one of R¹⁰ or R¹² also corresponds to H, while the other is chosen from:
Cl, F, OH, SH, CF₂H, CF₃, OR¹⁴ or SR¹⁴, preferably OH or OR¹⁴, in particular OH or OC₁₋₃-alkyl, preferably OH or OCH₃.

In this context, for compounds of group e) it is particularly preferable if compounds of the formula III are present in the form of their diastereomers having the relative configuration IIIa 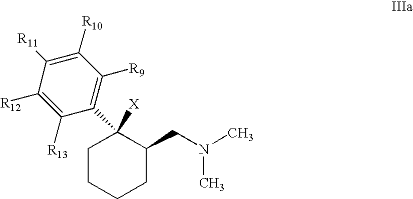
in particular are used in mixtures having a higher content of this diastereomer compared with the other diastereomer or as the pure diastereomer and/or
   if the compounds of the formula III are used in the form of the (+)-enantiomer, in particular in mixtures having a higher content of the (+)-enantiomer compared with the (−)-enantiomer of a racemic compound or as the pure (+)-enantiomer.

In this context, it is particularly preferable if compound A chosen from the following group is used:
(+)-(1R,2R)-3-(2-dimethylaminomethyl-1-fluoro-cyclohexyl)-phenol,
(+)-(1S,2S)-3-(2-dimethylaminomethyl-cyclohexyl)-phenol or
(−)-(1R,2R)-3-(2-dimethylaminomethyl-cyclohexyl)-phenol,
   preferably as the hydrochloride

For a particularly preferred use, the compound B is chosen from:
darifenacin, duloxetine, oxybutynin or tolterodine,
is preferably chosen from
duloxetine, oxybutynin or tolterodine,
is preferably chosen from
oxybutynin or tolterodine.

For another particularly preferred use, the compound B is chosen from:
venlafaxine, fesoterodine, solifenacin (YM905), cizolirtine or resiniferatoxin.

Even if the uses according to the invention show only a low degree of side effects, it may also be advantageous, for example to avoid certain forms of dependency, also to use morphine antagonists, in particular naloxone, naltrexone and/or levallorphan, in addition to the combination of the compounds A and B.

The invention also provides an active compound combination of at least one of the compounds A and at least one of the compounds B, where compound A is chosen from:
Group a) comprising:
tramadol, O-demethyltramadol or O-demethyl-N-mono-demethyl-tramadol as the free base or acid and/or in the form of physiologically acceptable salts, in particular in the form of their physiologically acceptable acidic and basic salts or salts with cations and bases or with anions and acids; in the form of the enantiomers, diastereomers, in particular mixtures of their enantiomers or diastereomers, or an individual enantiomer or diastereomer;
Group b) comprising:
   codeine
   dextropropoxyphene
   dihydrocodeine
   diphenoxylate
   ethylmorphine
   meptazinol
   nalbuphine
   pethidine (meperidine)
   tilidine
   tramadol
   viminol
   butorphanol
   dextromoramide
   dezocine
   diacetylmorphine (heroin)
   hydrocodone
   hydromorphone
   ketobemidone
   levomethadone
   levomethadyl acetate (1-α-acetylmethadol (LAAM))
   levorphanol
   morphine
   nalorphine
   oxycodone
   pentazocine
   piritramide
   alfentanil
   buprenorphine
   etorphine
   fentanyl
   remifentanil
   sufentanil
as the free base or acid and/or in the form of physiologically acceptable salts, in particular in the form of their physiologically acceptable acidic and basic salts or salts with cations and bases or with anions and acids, optionally in the form of the enantiomers, diastereomers, in particular mixtures of their enantiomers or diastereomers, or an individual enantiomer or diastereomer;
Group c) comprising:
1-phenyl-3-dimethylamino-propane compounds according to the general formula I 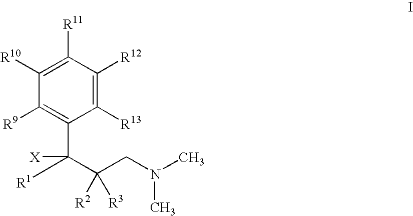
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted,
R¹ is chosen from C₁₋₄-alkyl, branched or unbranched, saturated or unsaturated, unsubstituted or mono- or polysubstituted,
R² and R³ in each case independently of one another are chosen from H or C₁₋₄-alkyl, branched or unbranched, saturated or unsaturated, unsubstituted or mono- or polysubstituted, or
R² and R³ together form a saturated C₄₋₇-cycloalkyl radical, unsubstituted or mono- or polysubstituted,
R⁹ to R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄-(C₁₋₃-alkyl), CO(C₁₋₅-alkyl), CO_CHR¹⁷—NHR¹⁸, C₀-C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring,
as the free base or acid and/or in the form of physiologically acceptable salts, in particular in the form of their physiologically acceptable acidic and basic salts or salts with cations and bases or with anions and acids; in the form of the enantiomers, diastereomers, in particular mixtures of their enantiomers or diastereomers, or an individual enantiomer or diastereomer;
Group d) comprising:
substituted 6-dimethylaminomethyl-1-phenylcyclohexane compounds according to the general formula II 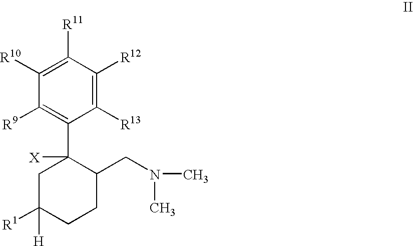
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted,
R¹ is chosen from C₁₋₄-alkyl, benzyl, CF₃, OH, OCH₂—C₆H₅, O—C₁₋₄-alkyl, Cl or F and R⁹ to R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄—(C₁₋₃-alkyl), CO(C5 s-alkyl), CO—CHR¹⁷—NHR¹⁸, CO—C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring, as the free base or acid and/or in the form of physiologically acceptable salts, in particular in the form of their physiologically acceptable acidic and basic salts or salts with cations and bases or with anions and acids; in the form of the enantiomers, diastereomers, in particular mixtures of their enantiomers or diastereomers, or an individual enantiomer or diastereomer;
and/or
Group e) comprising:
6-dimethylaminomethyl-1-phenyl-cyclohexane compounds according to the general formula III 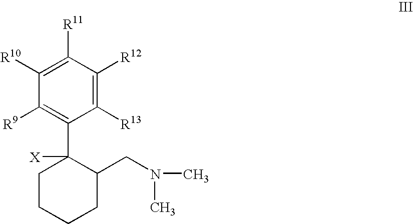
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted, and
R⁹ to R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₄-alkyl)₂, CO(OC₁₋₅-alkyl), CONH—C₆H₄—(C₁₋₃-alkyl), CO(C₁₋₅-alkyl), CO—CHR¹⁷—NHR¹⁸, CO_C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring,
with the proviso that if R⁹, R¹¹ and R¹³ correspond to H and one of R¹⁰ or R¹² corresponds to H and the other corresponds to OCH₃, X may not be OH,
as the free base or acid and/or in the form of physiologically acceptable salts, in particular in the form of their physiologically acceptable acidic and basic salts or salts with cations and bases or with anions and acids; in the form of the enantiomers, diastereomers, in particular mixtures of their enantiomers or diastereomers, or an individual enantiomer or diastereomer;
and with at least one of the compounds B chosen from
the antimuscarinics: atropine, oxybutynin, propiverine, propantheline, emepronium, trospium, tolterodine, darifenacin and α,α-diphenylacetic acid 4-(N-methylpiperidyl) ester, as well as duloxetine, imipramine and desmopressin, and
venlafaxine, fesoterodine, solifenacin (YM905), cizolirtine, resiniferatoxin, nitro-flurbiprofen, HCT1026, talnetant, TAK-637, SL 251039, R 450, Rec 15/3079, (−)-DDMS, NS-8 and/or DRP-001
as the free base or acid and/or in the form of physiologically acceptable salts, in particular in the form of their physiologically acceptable acidic and basic salts or salts with cations and bases or with anions and acids, optionally in the form of the enantiomers, diastereomers, in particular mixtures of their enantiomers or diastereomers, or an individual enantiomer or diastereomer.

Suitable salts in the context of this invention and in each of the medicaments described are salts of the particular active compound with inorganic or organic acids and/or a sugar substitute, such as saccharin, cyclamate or acesulfame. However, the hydrochloride is particularly preferred.

For the active compound combination, it is particularly preferable if the compound A in group a) is chosen from:
tramadol, (+)-tramadol, (+)—O-demethyltramadol or (+)—O-demethyl-N-mono-demethyl-tramadol, preferably tramadol or (+)-tramadol, in particular (+)-tramadol.

For the active compound combination, it is particularly preferable if compound A in group b) is chosen from:
codeine
dextropropoxyphene
dihydrocodeine
diphenoxylate
ethylmorphine
meptazinol
nalbuphine
pethidine (meperidine)
tilidine
viminol
butorphanol
dezocine
nalorphine
pentazocine
buprenorphine
   preferably
codeine
dextropropoxyphene
dihydrocodeine
meptazinol
nalbuphine
tilidine
buprenorphine

For the active compound combination, it is particularly preferable if the compound A in group c) is chosen from compounds according to formula I for which:
X is chosen from
OH, F, Cl, OC(O)CH₃ or H, preferably OH, F, OC(O)CH₃ or H, and/or
R¹ is chosen from
C₁₋₄-alkyl, saturated and unsubstituted, branched or unbranched; preferably CH₃, C₂H₅, C₄H₉ or t-butyl, in particular CH₃ or C₂H₅, and/or
R² and R³ independently of one another are chosen from
H or C₁₋₄-alkyl, saturated and unsubstituted, branched or unbranched; preferably H, CH₃, C₂H₅, i-propyl or t-butyl, in particular H or CH₃, preferably R³═H, or
R² and R³ together form a C₅₋₆-cycloalkyl radical, saturated or unsaturated, unsubstituted or mono- or polysubstituted, preferably saturated and unsubstituted, in particular cyclohexyl, and/or
R⁹ to R¹³, where 3 or 4 of the radicals R⁹ to R¹³ must correspond to H, independently of one another are chosen from
H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
preferably H, Cl, F, OH, CF₂H, CF₃, OCH₃ or SCH₃
or R¹² and R¹¹ form a 3,4-OCH═CH ring in particular
if R⁹, R¹¹ and R¹³ correspond to H, one of R¹⁰ or R¹² also corresponds to H, while the other is chosen from:
Cl, F, OH, CF₂H, CF₃, OR¹⁴ or SR¹⁴, preferably OH, CF₂H, OCH₃ or SCH₃ or
if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F, preferably Cl, or
if R⁹, R¹⁰, R¹² and R¹³ correspond to H, R¹¹ is chosen from CF₃, CF₂H, Cl or F, preferably F, or
if R¹⁰, R¹¹ and R¹² correspond to H, one of R⁹ or R¹³ also corresponds to H, while the other is chosen from OH, OC₂H₅ or OC₃H₇.

In this context, for compounds of group c) it is particularly preferable if the compounds of the formula I where R³═H are present in the form of the diastereomers having the relative configuration Ia 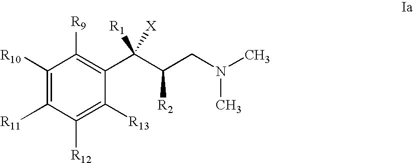
in particular in mixtures having a higher content of this diastereomer compared with the other diastereomer or as the pure diastereomer and/or
if the compounds of the formula I are present in the form of the (+)-enantiomer, in particular in mixtures having a higher content of the (+)-enantiomer compared with the (−)-enantiomer of a racemic compound or as the pure (+)-enantiomer.

In this context, it is particularly preferable if compound A is chosen from the following group:
(2RS,3RS)-1-dimethylamino-3-(3-methoxy-phenyl)-2-methyl-pentan-3-ol,
(+)-(2R,3R)-1-dimethylamino-3-(3-methoxy-phenyl)-2-methyl-pentan-3-ol,
(2RS,3RS)-3-(3,4-dichlorophenyl)-1-dimethylamino-2-methyl-pentan-3-ol,
(2RS,3RS)-3-(3-difluoromethyl-phenyl)-1-dimethylamino-2-methyl-pentan-3-ol,
(2RS,3RS)-1-dimethylamino-2-methyl-3-(3-methylsulfanyl-phenyl)-pentan-3-ol,
(3RS)-1-dimethylamino-3-(3-methoxy-phenyl)-4,4-dimethyl-pentan-3-ol,
(2RS,3RS)-3-(3-dimethylamino-1-ethyl-1-hydroxy-2-methyl-propyl)-phenol,
(1RS,2RS)-3-(3-dimethylamino-1-hydroxy-1,2-dimethyl-propyl)-phenol,
(+)-(1R,2R)-3-(3-dimethylamino-1-hydroxy-1,2-dimethyl-propyl)-phenol,
(+)-(1R,2R)-3-(3-dimethylamino-1-hydroxy-1,2-dimethyl-propyl)-phenol,
(−)-(1R,2R)-3-(3-dimethylamino-1-ethyl-2-methyl-propyl)-phenol,
(+)-(1R,2R)-acetic acid 3-dimethylamino-1-ethyl-1-(3-methoxy-phenyl)-2-methyl-propyl ester,
(1RS)-1-(1-dimethylaminomethyl-cyclohexyl)-1-(3-methoxy-phenyl)-propan-1-ol,
(2RS,3RS)-3-(4-chlorophenyl)-1-dimethylamino-2-methyl-pentan-3-ol,
(+)-(2R,3R)-3-(3-dimethylamino-1-ethyl-1-hydroxy-2-methyl-propyl-phenol,
(2RS,3RS)-4-dimethylamino-2-(3-methoxy-phenyl)-3-methyl-butan-2-ol and
(+)-(2R,3R)-4-dimethylamino-2-(3-methoxy-phenyl)-3-methyl-butan-2-ol,
   preferably as the hydrochloride.

For the active compound combination, it is particularly preferable if the compound A in group d) is chosen from compounds according to formula II for which:
X is chosen from
OH, F, Cl, OC(O)CH₃ or H, preferably OH, F or H, in particular OH, and/or
R¹ is chosen from
C₁₋₄-alkyl, CF₃, OH, O—C₁₋₄-alkyl, Cl or F, preferably OH, CF₃ or CH₃, and/or
R⁹ to R¹³, where 3 or 4 of the radicals R⁹ to R¹³ must correspond to H, independently of one another are chosen from
H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
preferably H, Cl, F, OH, CF₂H, CF₃, OCH₃ or SCH₃
or R¹² and R¹¹ form a 3,4-OCH═CH ring
in particular
if R⁹, R¹¹ and R¹³ correspond to H, one of R¹⁰ or R¹² also corresponds to H, while the other is chosen from:
Cl, F, OH, CF₂H, CF₃, OR¹⁴ or SR¹⁴, preferably OH, CF₂H, OR¹⁴ or SCH₃, in particular OH or OC₁₋₃-alkyl, preferably OH or OCH₃, or
if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F, preferably Cl, or
if R⁹, R¹⁰, R¹² and R¹³ correspond to H, R¹¹ is chosen from CF₃, CF₂H, Cl or F, preferably F, or
if R¹⁰, R¹¹ and R¹² correspond to H, one of R⁹ or R¹³ also corresponds to H, while the other is chosen from OH, OC₂H₅ or OC₃H₇.
very particularly preferably
if R⁹, R¹¹ and R¹³ correspond to H, one of R¹⁰ or R¹¹ also corresponds to H, while the other is chosen from:
Cl, F, OH, SH, CF₂H, CF₃, OR¹⁴ or SR¹⁴, preferably OH or OR¹⁴, in particular OH or OC₁₋₃-alkyl, preferably OH or OCH₃.

In this context, for compounds of group d) it is particularly preferable if the compounds of the formula II are present in the form of the diastereomers having the relative configuration IIa 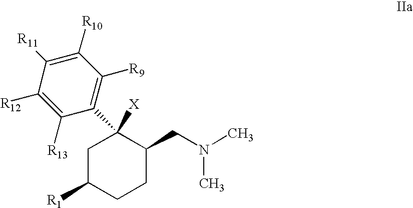
in particular in mixtures having a higher content of this diastereomer compared with the other diastereomer or as the pure diastereomer and/or
if the compounds of the formula II are present in the form of the (+)-enantiomer, in particular in mixtures having a higher content of the (+)-enantiomer compared with the (−)-enantiomer of a racemic compound or as the pure (+)-enantiomer.

In this context, it is particularly preferable if compound A is chosen from the following group:
(1RS,3RS,6RS)-6-dimethylaminomethyl-1-(3-methoxy-phenyl)-cyclohexane-1,3-diol,
(+)-(1R,3R,6R)-6-dimethylaminomethyl-1-(3-methoxy-phenyl)-cyclohexane-1,3-diol,
(1RS,3RS,6RS)-6-dimethylaminomethyl-1-(3-hydroxy-phenyl)-cyclohexane-1,3-diol,
(1RS,3 SR,6RS)-6-dimethylaminomethyl-1-(3-methoxy-phenyl)-cyclohexane-1,3-diol,
(+)-(1R,2R,5S)-3-(2-dimethylaminomethyl-1-hydroxy-5-methyl-cyclohexyl)-phenol or
(1RS,2RS,5RS)-3-(2-dimethylaminomethyl-1-hydroxy-5-trifluoromethyl-cyclohexyl)-phenol,
   preferably as the hydrochloride.

For the active compound combination, it is particularly preferable if the compound A in group e) is chosen from compounds according to formula III for which:
X is chosen from
OH, F, Cl, OC(O)CH₃ or H, preferably OH, F or H, in particular F or H, and/or
R⁹ to R¹³, where 3 or 4 of the radicals R⁹ to R¹³ must correspond to H, independently of one another are chosen from
H, Cl, F, OH, CF₂H, CF₃ or C₁₋₄-alkyl, saturated and unsubstituted, branched or unbranched; OR¹⁴ or SR¹⁴ , where R¹⁴ is chosen from C₁₋₃-alkyl, saturated and unsubstituted, branched or unbranched;
preferably H, Cl, F, OH, CF₂H, CF₃, OCH₃ or SCH₃
or R¹² and R¹¹ form a 3,4-OCH═CH ring
in particular characterized in that
if R⁹, R¹¹ and R¹³ correspond to H, one of R¹⁰ or R¹² also corresponds to H, while the other is chosen from:
Cl, F, OH, CF₂H, CF₃, OR¹⁴ or SR¹⁴, preferably OH, CF₂H, OR¹⁴ or SCH₃, in particular OH or OC₁₋₃-alkyl, preferably OH or OCH₃, or
if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F, preferably Cl, or
if R⁹, R¹⁰, R¹² and R¹³ correspond to H, R¹¹ is chosen from CF₃, CF₂H, Cl or F, preferably F, or
if R¹⁰, R¹¹ and R¹² correspond to H, one of R⁹ or R¹³ also corresponds to H, while the other is chosen from OH, OC₂H₅ or OC₃H₇;
very particularly preferably
if R⁹, R¹¹ and R¹³ correspond to H, one of R¹⁰ or R¹² also corresponds to H, while the other is chosen from:
Cl, F, OH, SH, CF₂H, CF₃, OR¹⁴ or SR¹⁴, preferably OH or OR¹⁴, in particular OH or OC₁₋₃-alkyl, preferably OH or OCH₃.

In this context, for compounds of group e) it is particularly preferable if the compounds of the formula III are present in the form of their diastereomers having the relative configuration IIIa 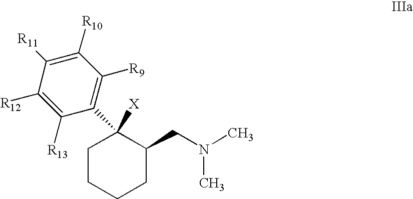
in particular in mixtures having a higher content of this diastereomer compared with the other diastereomer or as the pure diastereomer and/or
if the compounds of the formula III are present in the form of the (+)-enantiomer, in particular in mixtures having a higher content of the (+)-enantiomer compared with the (−)-enantiomer of a racemic compound or as the pure (+)-enantiomer.

In this context, it is particularly preferable if compound A is chosen from the following group:
(+)-(1R,2R)-3-(2-dimethylaminomethyl-1-fluoro-cyclohexyl)-phenol,
(+)-(1S,2S)-3-(2-dimethylaminomethyl-cyclohexyl)-phenol or
(−)-(1R,2R)-3-(2-dimethylaminomethyl-cyclohexyl)-phenol,
   preferably as the hydrochloride

In a generally particularly preferred form of the active compound combination according to the invention, the compound B is chosen from:
darifenacin, duloxetine, oxybutynin or tolterodine,
is preferably chosen from
duloxetine, oxybutynin or tolterodine,
is preferably chosen from oxybutynin or tolterodine.

For a particularly preferred form of the active compound combination according to the invention, the compound B is chosen from:
venlafaxine, fesoterodine, solifenacin (YM905), cizolirtine or resiniferatoxin.

The invention also provides a medicament, preferably for treatment of an increased urge to urinate and urinary incontinence, comprising an active compound combination according to the invention and optionally suitable additives and/or auxiliary substances.

Suitable additives and/or auxiliary substances in the context of this invention are all the substances known to the expert from the prior art for achieving galenical formulations. The choice of these auxiliary substances and the amounts thereof to be employed depend on whether the medicament is to be administered orally, intravenously, intraperitoneally, intradermally, intramuscularly, intranasally, buccally or locally. Formulations in the form of tablets, chewable tablets, coated tablets, capsules, granules, drops, juices or syrups are suitable for oral administration, and solutions, suspensions, easily reconstitutable dry formulations and sprays are suitable for parenteral, topical and inhalatory administration. Suppositories for use in the rectum are a further possibility. The use in a depot in dissolved form, a carrier film or a patch, optionally with the addition of agents which promote penetration through the skin, are examples of suitable forms for percutaneous administration. Examples of auxiliary substances and additives for the oral administration forms are disintegrating agents, lubricants, binders, fillers, mould release agents, optionally solvents, flavourings, sugars, in particular carrier agents, diluents, dyestuffs, antioxidants etc. For suppositories, inter alia, waxes and fatty acid esters can be used, and for parental administration compositions carrier substances, preservatives, suspension auxiliaries etc. can be used. The amounts of active compound to be administered to patients vary as a function of the weight of the patient, the mode of administration and the severity of the disease. The compounds according to the invention can be released in a delayed manner from formulation forms which can be used orally, rectally or percutaneously. Corresponding sustained-release formulations, in particular in the form of a “once daily” preparation which has to be taken only once a day, are particularly preferred for the indication according to the invention.

Medicaments which comprise at least 0.05 to 90.0% of the active compound, in particular low active dosages, in order to avoid side effects or analgesic actions, are furthermore preferred. 0.1 to 5,000 mg/kg, in particular 1 to 500 mg/kg, preferably 2 to 250 mg/kg of body weight of at least one compound of the formula I are conventionally administered. However, the administration of 0.01-5 mg/kg, preferably 0.03 to 2 mg/kg, in particular 0.05 to 1 mg/kg, is also preferred and conventional.

Auxiliary substances can be, for example: water, ethanol, 2-propanol, glycerol, ethylene glycol, propylene glycol, polyethylene glycol, polypropylene glycol, glucose, fructose, lactose, sucrose, dextrose, molasses, starch, modified starch, gelatine, sorbitol, inositol, mannitol, microcrystalline cellulose, methylcellulose, carboxymethylcellulose, cellulose acetate, shellac, cetyl alcohol, polyvinylpyrrolidone, paraffins, waxes, naturally occurring and synthetic gums, gum acacia, alginates, dextran, saturated and unsaturated fatty acids, stearic acid, magnesium stearate, zinc stearate, glyceryl stearate, sodium lauryl sulfate, edible oils, sesame oil, coconut oil, groundnut oil, soya bean oil, lecithin, sodium lactate, polyoxyethylene and -propylene fatty acid esters, sorbitan fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talc, kaolin, pectin, crospovidone, agar and bentonite.

The medicaments and pharmaceutical compositions according to the invention are prepared with the aid of means, devices, methods and processes which are well-known in the prior art of pharmaceutical formulation, such as are described, for example, in “Remington's Pharmaceutical Sciences”, ed. A. R. Gennaro, 17th ed., Mack Publishing Company, Easton, Pa. (1985), in particular in part 8, chapter 76 to 93.

Thus e.g. for a solid formulation, such as a tablet, the active compound of the medicament can be granulated with a pharmaceutical carrier, e.g. conventional tablet constituents, such as maize starch, lactose, sucrose, sorbitol, talc, magnesium stearate, dicalcium phosphate or pharmaceutically acceptable gums, and pharmaceutical diluents, such as e.g. water, in order to form a solid composition which comprises the active compound in homogeneous distribution. Homogeneous distribution is understood here as meaning that the active compound is uniformly distributed over the entire composition, so that this can easily be divided into unit dose forms, such as tablets, pills or capsules, having the same activity. The solid composition is then divided into unit dose forms. The tablets or pills of the medicament according to the invention or of the compositions according to the invention can also be coated or compounded in another manner in order to provide a dose form with delayed release. Suitable coating compositions are, inter alia, polymeric acids and mixtures of polymeric acids with materials such as e.g. shellac, cetyl alcohol and/or cellulose acetate.

Even if the medicaments according to the invention show only a low degree of side effects, it may be advantageous, for example to avoid certain forms of dependency, to use morphine antagonists, in particular naloxone, naltrexone and/or levallorphan, in addition to the combination of the compounds A and B.

The invention also relates to a method for treatment of an increased urge to urinate and urinary incontinence, in which the active compound combination of compound A and compound B is used.

Certain embodiments of the present invention may be further understood by reference to the following specific examples. These examples and the terminology used herein is for the purpose of describing particular embodiments only and is not intended to be limiting.

### EXAMPLES

### Example 1

### List of the Substances Tested

A list of the compounds tested for their activity follows:

| Name | Cpd. no. |
|---|---|
| (2RS,3RS)-1-dimethylamino-3-(3-methoxy-phenyl)-2-methyl-pentan-3-ol, | 1 |
| hydrochloride | |
| (+)-(2R,3R)-1-dimethylamino-3-(3-methoxy-phenyl)-2-methyl-pentan-3-ol, | 2 |
| hydrochloride | |
| (2RS,3RS)-3-(3,4-dichlorophenyl)-1-dimethylamino-2-methyl-pentan-3-ol, | 3 |
| hydrochloride | |
| (2RS,3RS)-3-(3-difluoromethyl-phenyl)-1-dimethylamino-2-methyl-pentan- | 4 |
| 3-ol, hydrochloride | |
| (2RS,3RS)-1-dimethylamino-2-methyl-3-(3-methylsulfanyl-phenyl)-pentan- | 5 |
| 3-ol, hydrochloride | |
| (3RS)-1-dimethylamino-3-(3-methoxy-phenyl)-4,4-dimethyl-pentan-3-ol, | 6 |
| hydrochloride | |
| (2RS,3RS)-3-(3-dimethylamino-1-ethyl-1-hydroxy-2-methyl-propyl)- | 7 |
| phenol, hydrochloride | |
| (1RS,2RS)-3-(3-dimethylamino-1-hydroxy-1,2-dimethyl-propyl)-phenol, | 8 |
| hydrochloride | |
| (+)-(1R,2R)-3-(3-dimethylamino-1-hydroxy-1,2-dimethyl-propyl)-phenol, | 9 |
| hydrochloride | |
| (+)-(1R,2R)-3-(3-dimethylamino-1-hydroxy-1,2-dimethyl-propyl)-phenol, | 10 |
| hydrochloride | |
| (−)-(1R,2R)-3-(3-dimethylamino-1-ethyl-2-methyl-propyl)-phenol, | 11 |
| hydrochloride | |
| (+)-(1R,2R)-acetic acid 3-dimethylamino-1-ethyl-1-(3-methoxy-phenyl)-2- | 12 |
| methyl-propyl ester, hydrochloride | |
| (1RS)-1-(1-dimethylaminomethyl-cyclohexyl)-1-(3-methoxy-phenyl)- | 13 |
| propan-1-ol, hydrochloride | |
| (2RS,3RS)-3-(4-chlorophenyl)-1-dimethylamino-2-methyl-pentan-3-ol, | 14 |
| hydrochloride | |
| (+)-(1R,2R)-3-(2-dimethylaminomethyl-1-fluoro-cyclohexyl)-phenol, | 18 |
| hydrochloride | |
| (+)-(1S,2S)-3-(2-dimethylaminomethyl-cyclohexyl)-phenol, hydrochloride | 19 |
| (−)-(1R,2R)-3-(2-dimethylaminomethyl-cyclohexyl)-phenol, hydrochloride | 20 |
| rac-tramadol | 23 |
| (−)-(2S,3S)-1-dimethylamino-3-(3-methoxy-phenyl)-2-methyl-pentan-3-ol, | 21 |
| hydrochloride | |
| (1RS,3RS,6RS)-6-dimethylaminomethyl-1-(3-methoxy-phenyl)- | 24 |
| cyclohexane-1,3-diol, hydrochloride | |
| (+)-(1R,3R,6R)-6-dimethylaminomethyl-1-(3-methoxy-phenyl)- | 25 |
| cyclohexane-1,3-diol, hydrochloride | |
| (1RS,3RS,6RS)-6-dimethylaminomethyl-1-(3-hydroxy-phenyl)- | 26 |
| cyclohexane-1,3-diol, hydrochloride | |
| (1RS,3SR,6RS)-6-dimethylaminomethyl-1-(3-methoxy-phenyl)- | 27 |
| cyclohexane-1,3-diol, hydrochloride | |
| (+)-(1R,2R,5S)-3-(2-dimethylaminomethyl-1-hydroxy-5-methyl- | 28 |
| cyclohexyl)-phenol, hydrochloride | |
| (1RS,2RS,5RS)-3-(2-dimethylaminomethyl-1-hydroxy-5-trifluoromethyl- | 29 |
| cyclohexyl)-phenol, hydrochloride | |

### Example 2

### Cystometry Test System on Conscious Naive Rats

Cystometric studies were carried out on naive, female Sprague-Dawley rats in accordance with the method of Ishizuka et al. ((1997), Naunyn-Schmiedeberg'a Arch. Pharmacol. 355: 787-793). Three days after implantation of bladder and venous catheters, the animals were investigated in the freely mobile, conscious state. The bladder catheter was connected to a pressure transducer and an injection pump. The animals were placed in metabolism cages which allowed measurement of the volume of urine. Physiological saline solution was infused into the emptied bladder (10 ml/h) and the bladder pressure and micturition volume were recorded continuously. After a stabilization phase, a 20 minute phase, which was characterized by normal, reproducible micturition cycles, was recorded. The following parameters, inter alia, were determined:
threshold pressure (TP, bladder pressure immediately before micturition),
bladder capacity (BC, residual volume after preceding micturition plus volume of the infused solution during the filling phase),
intercontraction interval (ICI, the interval of time between micturitions).

An increase in the threshold pressure (TP) indicates an important therapeutic action on one of the indications according to the invention. The intercontraction interval (ICI) is also an important parameter for measuring the physiological activity of a substance in the treatment of urinary incontinence, as is the bladder capacity (BC). In this context, because of the very heterogeneous causes of the symptoms of this clinical picture, for an activity it is not necessary to influence all three parameters positively. It is therefore entirely sufficient if a positive action can be detected in only one of these parameters in order for it to be possible to employ a substance on urinary incontinence or an increased urge to urinate.

After three reproducible micturition cycles had been recorded as the pre-value, test substances 1 (1.0 mg/kg), 2 (0.1; 0.3 and 0.5 mg/kg), 21 (0.5 mg/kg), 7 (0.3 mg/kg), 8 (1.0 mg/kg), 9 (0.5 mg/kg) and 11 (0.5 mg/kg) were administered i.v. in the vehicle=0.9% NaCl and the action on the cystometric parameters was recorded for 90 to 120 minutes. At the action maximum the mean of 3 micturition cycles was determined and shown as a percentage change compared with the pre-value (table 1).

TABLE 1
Influencing of cystometric parameters by the test
substances (change relative to the pre-value [%]);
n corresponds to the number of test animals.

| | TP | BC | ICI |
|---|---|---|---|
| Compound: | threshold | bladder | intercontraction |
| (concentration) | pressure | capacity | interval |
| 1 | +94% ** | +31% *** | +42% |
| 1.0 mg/kg iv | | | |
| (n = 9) | | | |
| 2 | +28.5% ** | +7.8% | +15.6% |
| 0.1 mg/kg iv | +122% ** | +33% * | +28% * |
| (n = 5) | +77.5% ** | +20.6% * | +28.6% * |
| 0.3 mg/kg iv | | | |
| (n = 8) | | | |
| 0.5 mg/kg iv | | | |
| (n = 9 | | | |
| 21 | −1.1% | +3% | +10% |
| 0.5 mg/kg iv | | | |
| (n = 8) | | | |
| 7 | +95% ** | +32% * | +28% * |
| 0.3 mg/kg iv | | | |
| (n = 7) | | | |
| 8 | +60% ** | +7% | +14.4% |
| 1.0 mg/kg i | | | |
| (n = 8) | | | |
| 9 | +56% ** | +50% ** | +21% * |
| 0.5 mg/kg iv | | | |
| (n = 7) | | | |
| 11 | +9% | +11% | +22.6% |
| 0.5 mg/kg iv | | | |
| (n = 8) | | | |

Significance (Student T test):
* p < 0.05;
** p < 0.01;
*** p < 0.001.

The substances investigated show a positive action on bladder regulation and are therefore suitable for treatment of urinary incontinence.

It is found, inter alia, that of the enantiomers of the racemic compound 1, only the (+)-enantiomer (compound 2) is effectively active (and is therefore a particularly preferred compound of this invention), while the (−)-enantiomer (compound 21) does not contribute to the action.

Further experiments were undertaken with other compounds.

After three reproducible micturition cycles had been recorded as the pre-value, test substances 24 (1.0; 3.0; 5.0 mg/kg), 25 (1.5 mg/kg) and 26 (3.0 mg/kg) were administered i.v. in the vehicle=0.9% NaCl and the action on the cystometric parameters was recorded for 90 to 120 minutes. At the action maximum the mean of 3 micturition cycles was determined and shown as a percentage change compared with the pre-value (table 2).

TABLE 2
Table 2: Influencing of cystometric parameters by the
test substances (change relative to the pre-value [%]);
n corresponds to the number of test animals.

| | TP | BC | ICI |
|---|---|---|---|
| Compound: | threshold | bladder | interconnection |
| (concentration) | pressure | capacity | interval |
| 24 | +44.0% *** | −8.0% | −15% ** |
| 1.0 mg/kg iv | +94.0% ** | −16.0% * | −16% * |
| (n = 7) | +69.0% * | −26.0% * | −21.2% |
| 3.0 mg/kg iv | | | |
| (n = 8) | | | |
| 5.0 mg/kg iv | | | |
| (n = 8) | | | |
| 25 | +62.0% * | −14.0% * | −9.0 * |
| 1.5 mg/kg iv | | | |
| (n = 8) | | | |
| 26 | +86.0% *** | +29.0% * | +27.0% * |
| 3.0 mg/kg iv | | | |
| (n = 7) | | | |

Significance (Student T test):
* p < 0.05;
** p < 0.01;
*** p < 0.001.

The substances investigated show a positive action on bladder regulation and are therefore suitable for treatment of urinary incontinence.

### Example 3

### Cystometry Test System on Narcotized Naive Rats

The cystometric investigation on naive female rats was carried out in accordance with the method of Kimura et al. (Kimura et al., 1996, Int. J. Urol. 3: 218-227). The abdomen of narcotized, ventilated rats is opened up and the ureter is ligated. The urine is drained from the kidneys. A catheter is inserted into the bladder and fixed. Saline is infused into the bladder via this by means of an infusion pump, until the bladder shows rhythmic spontaneous activity in the form of contractions, which can be recorded via a connected pressure transducer. After stable starting values have been reached, the test substance is administered i.v. in a cumulative manner. Influencing of the bladder function manifests itself via suppression of the spontaneous contractions. In this context, the absence of contractions over a period of 10 min is a parameter for the suppression.

With all the substances listed here, a suppression of the spontaneous contractions was measurable in the rats, table 3 showing the mean of the lowest dose of at least 2 experiments at which for the first time contractions were absent over a period of 10 minutes.

TABLE 3
Table 3: (n corresponds to the number
of experiments included in the value)

| Cpd. no. | Lowest dose (mg/kg) | |
|---|---|---|
| 3 | 23.3 | (n = 3) |
| 4 | 1.7 | (n = 3) |
| 5 | 2.3 | (n = 3) |
| 6 | 16.7 | (n = 3) |
| 10 | 0.2 | (n = 3) |
| 12 | 30.0 | (n = 3) |
| 13 | 20.0 | (n = 2) |
| 14 | 20.0 | (n = 2) |

The substances investigated show a positive action on bladder regulation and are therefore suitable for treatment of urinary incontinence.

Further experiments were undertaken with other compounds.

With all the substances listed here, a suppression of the spontaneous contractions was measurable in the rats, table 4 showing the mean of the lowest dose of at least 2 experiments at which for the first time contractions were absent over a period of 10 minutes.

TABLE 4
Table 4: (n corresponds to the number
of experiments included in the value)

| Cpd. no. | Lowest dose (mg/kg) | |
|---|---|---|
| 27 | 115 | (n = 2) |
| 28 | 16.7 | (n = 3) |
| 29 | 23.3 | (n = 3) |

The substances investigated show a positive action on bladder regulation and are therefore suitable for treatment of urinary incontinence.

Further experiments were undertaken with other compounds.

With all the substances listed here, a suppression of the spontaneous contractions was measurable in the rats, table 5 showing the mean of the lowest dose of at least 2 experiments at which for the first time contractions were absent over a period of 10 minutes.

TABLE 5
Table 5: (n corresponds to the number
of experiments included in the value)

| | |
|---|---|
| Cpd. no. | Lowest dose (mg/kg) |

| | | | |
|---|---|---|---|
| 18 | | 0.2 | (n = 3) |
| 19 | | 0.1 | (n = 3) |
| 20 | | 0.5 | (n = 3) |
| 23 | (tramadol) | 5.3 | (n = 3) |

The substances investigated show a positive action on bladder regulation and are therefore suitable for treatment of urinary incontinence and also appear to be superior to tramadol in this.

The following substances were furthermore tested, with the result shown in table 6:

With all the substances listed here, a suppression of the spontaneous contractions was measurable in the rats, table 6 showing the mean of the lowest dose of at least 3 independent experiments at which for the first time contractions were absent over a period of 10 minutes.

TABLE 6
Table 6: (n corresponds to the number
of experiments included in the value)

| Compound | Lowest dose (mg/kg) | |
|---|---|---|
| tilidine | 0.5 | (n = 3) |
| meptazinol | 1.0 | (n = 3) |
| codeine (phosphate) | 4.7 | (n = 3) |

The substances investigated show a positive action on bladder regulation and are therefore suitable for treatment of urinary incontinence.

### Example 4

### Cystometry Test System on Conscious Naive Rats

Cystometric studies were carried out on naive, female Sprague-Dawley rats in accordance with the method of Ishizuka et al. ((1997), Naunyn-Schmiedeberg's Arch. Pharmacol. 355: 787-793). Three days after implantation of bladder and venous catheters, the animals were investigated in the freely mobile, conscious state. The bladder catheter was connected to a pressure transducer and an injection pump. The animals were placed in metabolism cages which allowed measurement of the volume of urine. Physiological saline solution was infused into the emptied bladder (10 ml/h) and the bladder pressure and micturition volume were recorded continuously. After a stabilization phase, a 20 minute phase, which was characterized by normal, reproducible micturition cycles, was recorded. The following parameters, inter alia, were determined:
threshold pressure (TP, bladder pressure immediately before micturition),
bladder capacity (BC, residual volume after preceding micturition plus volume of the infused solution during the filling phase),
intercontraction interval (ICI, the interval of time between micturitions).

An increase in the threshold pressure (TP) indicates an important therapeutic action on one of the indications according to the invention. The intercontraction interval (ICI) is also an important parameter for measuring the physiological activity of a substance in the treatment of urinary incontinence, as is the bladder capacity (BC). In this context, because of the very heterogeneous causes of the symptoms of this clinical picture, for an activity it is not necessary to influence all three parameters positively. It is therefore entirely sufficient if a positive action can be detected in only one of these parameters in order for it to be possible to employ a substance on urinary incontinence, increased frequency of micturition or an increased urge to urinate.

After three reproducible micturition cycles had been recorded as the pre-value, 10 μg/kg buprenorphine were administered i.v. in the vehicle=0.9% NaCl and the action on the cystometric parameters was recorded for 90 to 120 minutes. At the action maximum the mean of 3 micturition cycles was determined and shown as a percentage change compared with the pre-value (table 7).

The concentration employed corresponds to the ED₅₀ in a known analgesia model for rats, the tail flick.

TABLE 7
Table 7: Influencing of cystometric parameters by buprenorphine(change
relative to the pre-value [%]); n corresponds to the number
of test animals employed in the study.

| | TP | BC | ICI |
|---|---|---|---|
| | threshold | bladder | intercontraction |
| Buprenorphine | pressure | capacity | interval |
| 0.01 mg/kg iv | +69.9% | +3.6% | +10.9% |
| (n = 6) | ** | | |

Significance (Student T test):
* p < 0.05;
** p < 0.01;
*** p < 0.001.

Buprenorphine shows a positive action on bladder regulation precisely on the TP and is therefore suitable in principle for treatment of urinary incontinence. Nevertheless, the concentration employed, which has an analgesic action, was evidently too high, since drip incontinence occurred in 2 of the 6 animals. At two lower concentrations, 0.001 mg/kg i.v. and 0.005 mg/kg i.v., an increase in the TP of +27.6% and +37.5% respectively occurred in n=6.

### Example 5

### Cystometry Test System on Conscious Damaged Rats

This model simulates the urgency incontinence in an animal model; the oxyhaemoglobin (OxyHb) employed induces a bladder hyperactivity.

Cystometric studies were carried out on naive, female Sprague-Dawley rats in accordance with the method of Pandita et al. (J. Urol. 2000, 164: 545-550)). Three days after implantation of bladder and venous catheters, the animals were investigated in the freely mobile, conscious state. The bladder catheter was connected to a pressure transducer and an injection pump. The animals were placed in metabolism cages which allowed measurement of the volume of urine. Physiological saline solution was infused into the emptied bladder (10 ml/h) and the bladder pressure and micturition volume were recorded continuously. After a stabilization phase, a 20 minute phase, which was characterized by normal, reproducible micturition cycles, was recorded. The following parameters, inter alia, were determined:
threshold pressure (TP, bladder pressure immediately before micturition),
bladder capacity (BC, residual volume after preceding micturition plus volume of the infused solution during the filling phase),
intercontraction interval (ICI, the interval of time between micturitions)
micturition pressure (MP, maximum bladder pressure during a micturition).

An increase in the threshold pressure (TP) indicates an important therapeutic action on one of the indications according to the invention. The intercontraction interval (ICI) is also an important parameter for measuring the physiological activity of a substance in the treatment of urinary incontinence, as is the bladder capacity (BC). In this context, because of the very heterogeneous causes of the symptoms of this clinical picture, for an activity it is not necessary to influence all the parameters positively. It is therefore entirely sufficient if a positive action can be detected in only one of these parameters in order for it to be possible to employ a substance on urinary incontinence, increased frequency of micturition or an increased urge to urinate.

After three reproducible micturition cycles had been recorded as the pre-value, 2.5×10⁻⁴ M oxyhemoglobin in the vehicle=0.9% NaCl were infused into the bladder. The action on the cystometric parameters was recorded for about 20 minutes. At the action maximum the mean of 3 micturition cycles was determined and shown as a percentage change compared with the pre-value (table 8). The treatment with oxyhaemoglobin induces a characteristic change in the cystometric parameters with an increase in the micturition pressure, a reduction in the bladder capacity and a reduction in the intercontraction interval. These changes mirror the changes found in patients with urgency incontinence.

The administration of 5 μg/kg buprenorphine i.v. in the vehicle=0.9% NaCl before the administration of oxyhemoglobin is capable of suppressing the changes induced by oxyhemoglobin and moreover also of inducing an increase in the threshold pressure (table 8).

TABLE 8
Table 8: Influencing of the cystometric parameters by oxyhaemoglobin (OxyHb)
with and without prior administration of buprenorphine. Average values with
standard deviations before (b) and after (a) use of the substances and the
change (diff.) in comparison with the pre-value [%] are stated; n
corresponds to the number of animals employed in the study.

| | MP | TP | BC | ICI |
|---|---|---|---|---|
| | micturition | threshold | bladder | intercontraction |
| | pressure | pressure | capacity | interval |
| | [cm H₂O] | [cm H₂O] | [ml] | [min] |
| OxyHb | | | | |
| 2.5 × 10⁻⁴ M iv | b: 59 ± 8 | b: 8.72 ± 1.31 | b: 0.92 ± 0.10 | b: 4.96 ± 0.33 |
| (n = 5) | a: 97 ± 5 | a: 9.84 ± 1.56 | a: 0.65 ± 0.06 | a: 3.33 ± 0.18 |
| | diff.: +64.4% | diff.: +12.8% | diff: −29.3% | diff: −32.9% |
| | ** | | ** | ** |
| OxyHb + | | | | |
| buprenorphine | | | | |
| OxyHb: | b: 54 ± 9 | b: 9.07 ± 1.29 | b: 1.19 ± 0.12 | b: 6.72 ± 0.73 |
| 2.5 × 10⁻⁴ M | a: 37 ± 8 | a: 14.28 ± 2.53 | a: 1.17 ± 0.13 | a: 6.70 ± 0.88 |
| buprenorphine: | diff.: −31.5% | diff.: +57.4% | diff: −1.7% | diff: −0.3% |
| 0.005 mg/kg iv | * | * | | |
| (n = 6) | | | | |

Significance (Student T test):
* p < 0.05;
** p < 0.01;
*** p < 0.001.

It can be seen that OxyHb clearly adversely influences the bladder parameters in the sense of urgency incontinence. This adverse influencing is eliminated by buprenorphine, and even improved. Thus, the micturition pressure falls significantly compared with the urgency incontinence induced by OxyHb and also compare with the untreated control. In this urgency incontinence model buprenorphine furthermore normalizes the intercontraction interval and the bladder capacity completely and moreover has the effect of a significant and clear increase in the threshold pressure.

Evidence is thus provided that buprenorphine, in particular in the area of urgency incontinence, for which the OxyHb model is the standard model, shows an outstanding action, and in particular also in the event of damage, that is to say in the case of disease.

### Example 6

### Parenteral Administration Form

20 g tramadol and 1 g venlafaxine are dissolved in 1 l water for injection purposes at room temperature and the solution is then adjusted to isotonic conditions by addition of NaCl.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations falling within the scope of the appended claims and equivalents thereof.

1. A pharmaceutical formulation comprising a combination of at least one compound selected from group A and at least one compound selected from group B, wherein group A consists of:
Group a) consisting of:
tramadol, O-demethyltramadol and O-demethyl-N-monodemethyl-tramadol;
Group b) consisting of:
codeine
dextropropoxyphene
dihydrocodeine
diphenoxylate
ethylmorphine
meptazinol
nalbuphine
pethidine (meperidine)
tilidine
tramadol
viminol
butorphanol
dextromoramide
dezocine
diacetylmorphine (heroin)
hydrocodone
hydromorphone
ketobemidone
levomethadone
levomethadyl acetate (1-α-acetylmethadol (LAAM))
levorphanol
morphine
nalorphine
oxycodone
pentazocine
piritramide
alfentanil
buprenorphine
etorphine
fentanyl
remifentanil and
sufentanil;
Group c) consisting of:
1-phenyl-3-dimethylamino-propane compounds corresponding to formula I 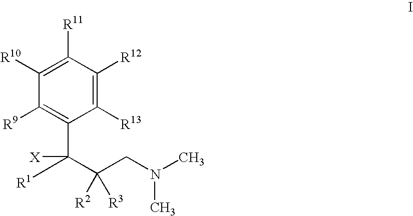
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted,
R¹ is chosen from C₁₋₄-alkyl, branched or unbranched, saturated or unsaturated, unsubstituted or mono- or polysubstituted,
R² and R³ in each case independently of one another are chosen from H or C₁₋₄-alkyl, branched or unbranched, saturated or unsaturated, unsubstituted or mono- or polysubstituted, or
R² and R³ together form a saturated C₄₋₇-cycloalkyl radical, unsubstituted or mono- or polysubstituted,
R⁹-R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄—(C₁₋₃-alkyl), CO(C₁₋₅-alkyl), CO—CHR¹⁷—NHR¹⁸, CO_C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring;
Group d) consisting of:
substituted 6-dimethylaminomethyl-1-phenylcyclohexane compounds corresponding to formula II 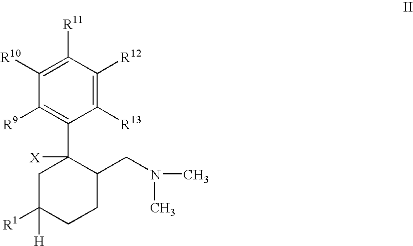
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted,
R¹ is chosen from C₁₋₄-alkyl, benzyl, CF₃, OH, OCH₂—C₆H₅, O—C₁₋₄-alkyl, Cl or F and
R⁹-R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C 14-alkyl)₂, CO(OC₁₋₅-alkyl), CONH—C₆H₄—(C₁₋₃-alkyl), CO(C₁₋₅-alkyl), CO—CHR¹⁷—NHR¹⁸, C₀-C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring and
Group e) consisting of:
6-dimethylaminomethyl-1-phenyl-cyclohexane compounds corresponding to formula III 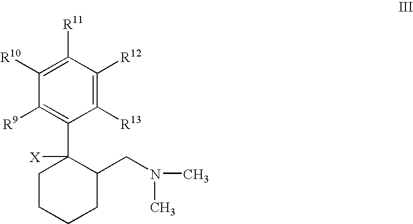
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted, and
R⁹-R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄—(C₁₋₃-alkyl), CO(C₁₋₅-alkyl), CO—CHR¹⁷—NHR¹⁸, CO—C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring,
with the proviso that if R⁹, R¹¹ and R¹³ correspond to H and one of R¹⁰ or R¹² corresponds to H and the other corresponds to OCH₃, X may not be OH, and
wherein group B consists of:
venlafaxine, fesoterodine, solifenacin (YM905), cizolirtine, resiniferatoxin, nitro-flurbiprofen, HCT1026, talnetant, TAK-637, SL 251039, R 450, Rec 15/3079, (−)-DDMS, NS-8 and DRP-001.

2. The pharmaceutical formulation of claim 1, wherein either or both of the compounds of Group A and Group B are present in the form of a salt with a physiologically tolerated acid.

3. The pharmaceutical formulation of claim 1, wherein either or both of the compounds of Group A and Group B are present in the form of an acid.

4. The pharmaceutical formulation of claim 1, wherein either or both of the compounds of Group A and Group B are present in the form of a free base.

5. The pharmaceutical formulation of claim 1, wherein either or both of the compounds of Group A and Group B are present in the form of a salt with a physiologically tolerated base.

6. The pharmaceutical formulation of claim 1, wherein either or both of the compounds of Group A and Group B are present in the form of an individual enantiomer or diastereoisomer.

7. The pharmaceutical formulation of claim 1, wherein either or both of the compounds of Group A and Group B are present in the form of a mixture of stereoisomers.

8. The pharmaceutical formulation of claim 1, wherein the compound A in group a) is selected from:
tramadol, (+)-tramadol, (+)—O-demethyltramadol and (+)—O-demethyl-N-mono-demethyl-tramadol.

9. The pharmaceutical formulation of claim 1, wherein the compound A in group a) is (+)-tramadol.

10. The pharmaceutical formulation of claim 1, wherein the compound A in group b) is chosen from:
codeine
dextropropoxyphene
dihydrocodeine
diphenoxylate
ethylmorphine
meptazinol
nalbuphine
pethidine (meperidine)
tilidine
viminol
butorphanol
dezocine
nalorphine
pentazocine and
buprenorphine.

11. The pharmaceutical formulation of claim 1, wherein the compound A in group b) is chosen from:
codeine
dextropropoxyphene
dihydrocodeine
meptazinol
nalbuphine
tilidine and
buprenorphine.

12. The pharmaceutical formulation of claim 1, wherein the compound A in group c) is chosen from compounds according to formula I for which:
X is chosen from OH, F, Cl, OC(O)CH₃ or H;
R¹ is chosen from C₁₋₄-alkyl, saturated and unsubstituted, branched or unbranched;
R² and R³ independently of one another are chosen from H or C₁₋₄-alkyl, saturated and unsubstituted, branched or unbranched; or
R² and R³ together form a C₅₋₆-cycloalkyl radical, saturated or unsaturated, unsubstituted or mono- or polysubstituted;
R⁹-R¹³, where 3 or 4 of the radicals R⁹-R¹³ must correspond to H, independently of one another are chosen from H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
or R¹² and R¹¹ form a 3,4-OCH═CH ring;
or if R⁹, R¹¹ and R¹³ correspond to H, one of R¹⁰ or R¹² also corresponds to H, while the other is chosen from: Cl, F, OH, CF₂H, CF₃, OR¹⁴ or SR¹⁴;
or if R⁹ and R¹³ correspond to H and R¹¹ corresponds to OH, OCH₃, Cl or F, one of R¹⁰ or R¹² also corresponds to H, while the other corresponds to OH, OCH₃, Cl or F;
or if R⁹, R¹⁰, R¹² and R¹³ correspond to H, R¹¹ is chosen from CF₃, CF₂H, Cl or F;
or if R¹⁰, R¹¹ and R¹² correspond to H, one of R⁹ or R¹³ also corresponds to H, while the other is chosen from: OH, OC₂H₅ or OC₃H₇.

13. The pharmaceutical formulation of claim 12, wherein compounds corresponding to formula I where R³═H are present in the form of the diastereomers having the relative configuration Ia 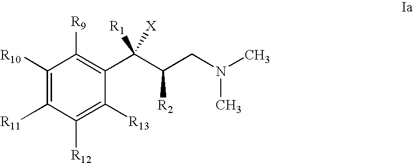
or
compounds corresponding to formula I are present in the form of the (+)-enantiomer.

14. The pharmaceutical formulation of claim 13, wherein compounds corresponding to formula I where R³═H are present in the form of the diastereomers having the relative configuration Ia in a greater amount than the other diastereomer or the pure diasteromer having the relative configuration Ia is provided or
compounds corresponding to formula I are present in the form of the (+)-enantiomer, said formulation having a higher content of the (+)-enantiomer compared with the (−)-enantiomer or said formulation having the pure (+)-enantiomer.

15. The pharmaceutical formulation of claim 12, wherein compound A is selected from the group consisting of:
(2RS,3RS)-1-dimethylamino-3-(3-methoxy-phenyl)-2-methyl-pentan-3-ol,
(+)-(2R,3R)-1-dimethylamino-3-(3-methoxy-phenyl)-2-methyl-pentan-3-ol,
(2RS,3RS)-3-(3,4-dichlorophenyl)-1-dimethylamino-2-methyl-pentan-3-ol,
(2RS,3RS)-3-(3-difluoromethyl-phenyl)-1-dimethylamino-2-methyl-pentan-3-ol,
(2RS,3RS)-1-dimethylamino-2-methyl-3-(3-methylsulfanyl-phenyl)-pentan-3-ol,
(3RS)-1-dimethylamino-3-(3-methoxy-phenyl)-4,4-dimethyl-pentan-3-ol,
(2RS,3RS)-3-(3-dimethylamino-1-ethyl-1-hydroxy-2-methyl-propyl)-phenol,
(1RS,2RS)-3-(3-dimethylamino-1-hydroxy-1,2-dimethyl-propyl)-phenol,
(+)-(1R,2R)-3-(3-dimethylamino-1-hydroxy-1,2-dimethyl-propyl)-phenol,
(+)-(1R,2R)-3-(3-dimethylamino-1-hydroxy-1,2-dimethyl-propyl)-phenol,
(−)-(1R,2R)-3-(3-dimethylamino-1-ethyl-2-methyl-propyl)-phenol,
(+)-(1R,2R)-acetic acid 3-dimethylamino-1-ethyl-1-(3-methoxy-phenyl)-2-methyl-propyl ester,
(1RS)-1-(1-dimethylaminomethyl-cyclohexyl)-1-(3-methoxy-phenyl)-propan-1-ol,
(2RS,3RS)-3-(4-chlorophenyl)-1-dimethylamino-2-methyl-pentan-3-ol,
(+)-(2R,3R)-3-(3-dimethylamino-1-ethyl-1-hydroxy-2-methyl-propyl-phenol,
(2RS,3RS)-4-dimethylamino-2-(3-methoxy-phenyl)-3-methyl-butan-2-ol and
(+)-(2R,3R)-4-dimethylamino-2-(3-methoxy-phenyl)-3-methyl-butan-2-ol.

16. The pharmaceutical formulation of claim 15, wherein compound A is in the form of a hydrochloride.

17. The pharmaceutical formulation of claim 1, wherein the compound A in group d) is chosen from compounds corresponding to formula II for which:
X is chosen from OH, F, Cl, OC(O)CH₃ or H;
R¹ is chosen from C₁₋₄-alkyl, CF₃, OH, O—C₁₋₄-alkyl, Cl or F;
R⁹-R¹³, where 3 or 4 of the radicals R⁹ to R¹³ must correspond to H, independently of one another are chosen from H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
or R¹² and R¹¹ form a 3,4-OCH═CH ring
or if R⁹, R¹¹ and R¹³ correspond to H, one of R¹⁰ or R¹² also corresponds to H, while the other is chosen from Cl, F, OH, CF₂H, CF₃, OR¹⁴ or SR¹⁴;
or if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F;
or if R⁹, R¹⁰, R¹² and R¹³ correspond to H, R¹¹ is chosen from CF₃, CF₂H, Cl or F;
or if R¹⁰, R¹¹ and R¹² correspond to H, one of R⁹ or R¹³ also corresponds to H, while the other is chosen from OH, OC₂H₅ or OC₃H₇;
or if R⁹, R¹¹ and R¹³ correspond to H, one of R¹⁰ or R¹² also corresponds to H, while the other is chosen from Cl, F, OH, SH, CF₂H, CF₃, OR¹⁴ or SR¹⁴.

18. A pharmaceutical formulation according to claim 17, wherein compounds corresponding to formula II are present in the form of the diastereomers having the relative configuration IIa 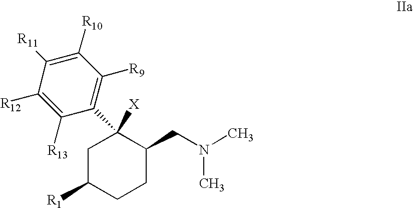
or
compounds corresponding to formula II are present in the form of the (+)-enantiomer.

19. A pharmaceutical formulation according to claim 18, wherein compounds corresponding to formula II are present in the form of the diastereomers having the relative configuration IIa in a greater amount than the other diastereomer or the pure diastereomer having the relative configuration IIa is provided or
compounds corresponding to formula II are present in the form of the (+)-enantiomer, said formulation having a higher content of the (+)-enantiomer compared with the (−)-enantiomer or said formulation having the pure (+)-enantiomer.

20. A pharmaceutical formulation according to claim 17, wherein compound A is selected from the group consisting of:
(1RS,3RS,6RS)-6-dimethylaminomethyl-1-(3-methoxy-phenyl)-cyclohexane-1,3-diol,
(+)-(1R,3R,6R)-6-dimethylaminomethyl-1-(3-methoxy-phenyl)-cyclohexane-1,3-diol,
(1RS,3RS,6RS)-6-dimethylaminomethyl-1-(3-hydroxy-phenyl)-cyclohexane-1,3-diol,
(1RS,3 SR,6RS)-6-dimethylaminomethyl-1-(3-methoxy-phenyl)-cyclohexane-1,3-diol,
(+)-(1R,2R,5S)-3-(2-dimethylaminomethyl-1-hydroxy-5-methyl-cyclohexyl)-phenol and
(1RS,2RS,5RS)-3-(2-dimethylaminomethyl-1-hydroxy-5-trifluoromethyl-cyclohexyl)-phenol.

21. A pharmaceutical formulation according to claim 20, wherein compound A is in the form of a hydrochloride.

22. A pharmaceutical formulation according to claim 1, wherein compound A in group e) is chosen from compounds corresponding to formula III for which:
X is chosen from OH, F, Cl, OC(O)CH₃ or H;
R⁹-R¹³, where 3 or 4 of the radicals R⁹ to R¹³ must correspond to H, independently of one another are chosen from H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
or R¹² and R¹¹ form a 3,4-OCH═CH ring;
or if R⁹, R¹¹ and R¹³ correspond to H, one of R¹⁰ or R¹² also corresponds to H, while the other is chosen from Cl, F, OH, CF₂H, CF₃, OR¹⁴ or SR¹⁴;
or if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F;
or if R⁹, R¹⁰, R¹² and R¹³ correspond to H, R¹¹ is chosen from CF₃, CF₂H, Cl or F;
or if R¹⁰, R¹¹ and R¹² correspond to H, one of R⁹ or R¹³ also corresponds to H, while the other is chosen from OH, OC₂H₅ or OC₃H₇;
or if R⁹, R¹¹ and R¹³ correspond to H, one of R¹⁰ or R¹² also corresponds to H, while the other is chosen from Cl, F, OH, SH, CF₂H, CF₃, OR¹⁴ or SR¹⁴.

23. A pharmaceutical formulation according to claim 22, wherein compounds corresponding to formula III are present in the form of their diastereomers having the relative configuration IIa 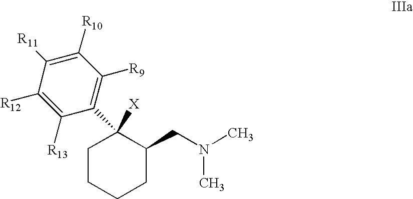
or
compounds corresponding to formula III are present in the form of the (+)-enantiomer.

24. A pharmaceutical formulation according to claim 23, wherein compounds corresponding to formula 1 ml are present in the form of the diastereomers having the relative configuration IIIa in a greater amount than the other diastereomer or the pure diastereomer having the relative configuration IIa is provided
or
compounds corresponding to formula III are present in the form of the (+)-enantiomer, said formulation having a higher content of the (+)-enantiomer compared with the (−)-enantiomer or said formulation having the pure (+)-enantiomer.

25. A composition of matter according to claim 22, wherein compound A is selected from the group consisting of:
(+)-(1R,2R)-3-(2-dimethylaminomethyl-1-fluoro-cyclohexyl)-phenol,
(+)-(1S,2S)-3-(2-dimethylaminomethyl-cyclohexyl)-phenol and
(−)-(1R,2R)-3-(2-dimethylaminomethyl-cyclohexyl)-phenol.

26. A composition of matter according to claim 25, wherein compound A is present in the form of a hydrochloride.

27. A composition of matter according to claim 1, wherein compound B is selected from the group consisting of:
fesoterodine, solifenacin (YM905), cizolirtine, resiniferatoxin and venlaxafine.

28. A pharmaceutical formulation comprising:
a composition of matter according to claim 1 and
a pharmaceutically acceptable auxiliary substance.

29. A method of treating increased urge to urinate or urinary incontinence in a mammal comprising administering to said mammal an effective amount of at least one compound selected from group A and at least one compound selected from group B, wherein group A consists of:
Group a) consisting of:
tramadol, O-demethyltramadol and O-demethyl-N-monodemethyl-tramadol;
Group b) consisting of:
codeine
dextropropoxyphene
dihydrocodeine
diphenoxylate
ethylmorphine
meptazinol
nalbuphine
pethidine (meperidine)
tilidine
tramadol
viminol
butorphanol
dextromoramide
dezocine
diacetylmorphine (heroin)
hydrocodone
hydromorphone
ketobemidone
levomethadone
levomethadyl acetate (1-α-acetylmethadol (LAAM))
levorphanol
morphine
nalorphine
oxycodone
pentazocine
piritramide
alfentanil
buprenorphine
etorphine
fentanyl
remifentanil and
sufentanil;
Group c) consisting of:
1-phenyl-3-dimethylamino-propane compounds corresponding to formula I 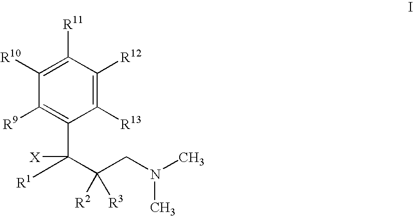
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted,
R¹ is chosen from C₁₋₄-alkyl, branched or unbranched, saturated or unsaturated, unsubstituted or mono- or polysubstituted,
R² and R³ in each case independently of one another are chosen from H or C₁₋₄-alkyl, branched or unbranched, saturated or unsaturated, unsubstituted or mono- or polysubstituted, or
R² and R³ together form a saturated C₄₋₇-cycloalkyl radical, unsubstituted or mono- or polysubstituted,
R⁹-R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄—(C₁₋₃-alkyl), CO(C₁₋₅-alkyl), CO—CHR¹⁷—NHR¹⁸, CO—C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring;
Group d) consisting of:
substituted 6-dimethylaminomethyl-1-phenylcyclohexane compounds corresponding to formula II 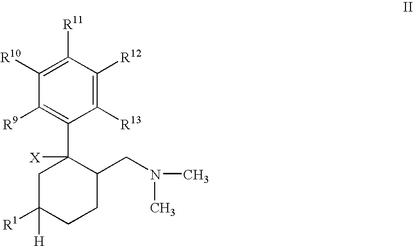
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted,
R¹ is chosen from C₁₋₄-alkyl, benzyl, CF₃, OH, OCH₂—C₆H₅, O—C₁₋₄-alkyl, Cl or F and
R⁹-R¹³ in each case independently of one another are chosen from H, F, Cl, Br, I, CH₂F, CHF₂, CF₃, OH, SH, OR¹⁴, OCF₃, SR¹⁴, N⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄—(C₁₋₃-alkyl), CO(C₁₋₅-alkyl), CO—CHR¹⁷—NHR¹⁸, CO—C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring and
Group e) consisting of:
6-dimethylaminomethyl-1-phenyl-cyclohexane compounds corresponding to formula III 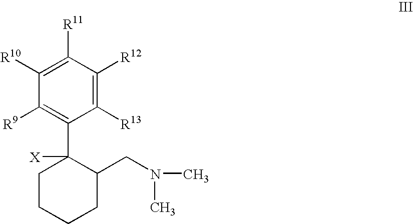
wherein
X is chosen from OH, F, Cl, H or OC(O)R⁷, where R⁷ is chosen from C₁₋₃-alkyl, branched or unbranched, saturated or unsaturated, unsubstituted or mono- or polysubstituted, and
R⁹-R¹³ in each case independently of one another are chosen from H, F, Cl, Br, I, CH₂F, CHF₂, CF₃, OH, SH, OR¹⁴, OCF₃, SR¹⁴, NR¹⁷R¹⁸, SOCH₃, SOCF₃; SO₂CH₃, SO₂CF₃, CN, COOR¹⁴, NO₂, CONR¹⁷R¹⁸; C₁₋₆-alkyl, branched or unbranched, saturated or unsaturated, unsubstituted or mono- or polysubstituted; phenyl, unsubstituted or mono- or polysubstituted;
where R¹⁴ is chosen from C₁₋₆-alkyl; pyridyl, thienyl, thiazolyl, phenyl, benzyl or phenethyl, in each case unsubstituted or mono- or polysubstituted; PO(O—C₁₋₄-alkyl)₂, CO(OC₁₋₅-alkyl), CONH—C₆H₄—(C₁₋₃-alkyl), CO(C₁₋₅-alkyl), CO—CHR¹⁷—NHR¹¹, CO—C₆H₄—R¹⁵, where R¹⁵ is ortho-OCOC₁₋₃-alkyl or meta- or para-CH₂N(R¹⁶)₂, where R¹⁶ is C₁₋₄-alkyl or 4-morpholino, wherein in the radicals R¹⁴, R¹⁵ and R¹⁶ the alkyl groups can be branched or unbranched, saturated or unsaturated, unsubstituted or mono- or polysubstituted;
where R¹⁷ and R¹⁸ in each case independently of one another are chosen from H; C₁₋₆-alkyl, branched or unbranched, saturated or unsaturated, unsubstituted or mono- or polysubstituted; phenyl, benzyl or phenethyl, in each case unsubstituted or mono- or polysubstituted, or
R⁹ and R¹⁰ or R¹⁰ and R¹¹ together form an OCH₂O, OCH₂CH₂O, OCH═CH, CH═CHO, CH═C(CH₃)O, OC(CH₃)═CH, (CH₂)₄ or OCH═CHO ring,
with the proviso that if R⁹, R¹¹ and R¹³ correspond to H and one of R¹⁰ or R¹² corresponds to H and the other corresponds to OCH₃, X may not be OH, and
wherein group B consists of:
venlafaxine, fesoterodine, solifenacin (YM905), cizolirtine, resiniferatoxin, nitro-flurbiprofen, HCT1026, talnetant, TAK-637, SL 251039, R 450, Rec 15/3079, (−)-DDMS, NS-8 and DRP-001.

30. The method of claim 29, wherein either or both of the compounds of Group A and Group B are present in the form of a salt with a physiologically tolerated acid.

31. The method of claim 29, wherein either or both of the compounds of Group A and Group B are present in the form of an acid.

32. The method of claim 29, wherein either or both of the compounds of Group A and Group B are present in the form of a free base.

33. The method of claim 29, wherein either or both of the compounds of Group A and Group B are present in the form of a salt with a physiologically tolerated base.

34. The method of claim 29, wherein either or both of the compounds of Group A and Group B are present in the form of an individual enantiomer or diastereoisomer.

35. The method of claim 29, wherein either or both of the compounds of Group A and Group B are present in the form of a mixture of stereoisomers.

36. The method of claim 29, wherein the compound A in group a) is selected from:
tramadol, (+)-tramadol, (+)—O-demethyltramadol and (+)—O-demethyl-N-mono-demethyl-tramadol.

37. The method of claim 29, wherein the compound A in group a) is (+)-tramadol.

38. The method of claim 29, wherein the compound A in group b) is chosen from:
codeine
dextropropoxyphene
dihydrocodeine
diphenoxylate
ethylmorphine
meptazinol
nalbuphine
pethidine (meperidine)
tilidine
viminol
butorphanol
dezocine
nalorphine
pentazocine and
buprenorphine.

39. The method of claim 29, wherein the compound A in group b) is chosen from:
codeine
dextropropoxyphene
dihydrocodeine
meptazinol
nalbuphine
tilidine and
buprenorphine.

40. The method of claim 29, wherein the compound A in group c) is chosen from compounds according to formula I for which:
X is chosen from OH, F, Cl, OC(O)CH₃ or H;
R¹ is chosen from C₁₋₄-alkyl, saturated and unsubstituted, branched or unbranched;
R² and R³ independently of one another are chosen from H or C₁₋₄-alkyl, saturated and unsubstituted, branched or unbranched; or
R² and R³ together form a C₅₋₆-cycloalkyl radical, saturated or unsaturated, unsubstituted or mono- or polysubstituted;
R⁹-R¹³, where 3 or 4 of the radicals R⁹-R¹³ must correspond to H, independently of one another are chosen from H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
or R¹² and R¹¹ form a 3,4-OCH═CH ring;
or if R⁹, R¹¹ and R¹³ correspond to H, one of R¹⁰ or R¹² also corresponds to H, while the other is chosen from: Cl, F, OH, CF₂H, CF₃, OR¹⁴ or SR¹⁴;
or if R⁹ and R¹³ correspond to H and R¹¹ corresponds to OH, OCH₃, Cl or F, one of R¹⁰ or R¹² also corresponds to H, while the other corresponds to OH, OCH₃, Cl or F;
or if R⁹, R¹⁰, R¹² and R¹³ correspond to H, R¹¹ is chosen from CF₃, CF₂H, Cl or F;
or if R¹⁰, R¹¹ and R¹² correspond to H, one of R⁹ or R¹³ also corresponds to H, while the other is chosen from: OH, OC₂H₅ or OC₃H₇.

41. The method of claim 40, wherein compounds corresponding to formula I where R³═H are present in the form of the diastereomers having the relative configuration Ia 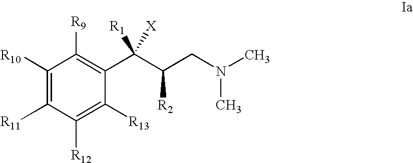
or
compounds corresponding to formula I are present in the form of the (+)-enantiomer.

42. The method of claim 41, wherein compounds corresponding to formula I where R³═H are present in the form of the diastereomers having the relative configuration Ia in a greater amount than the other diastereomer or the pure diasteromer having the relative configuration Ia is provided or
compounds corresponding to formula I are present in the form of the (+)-enantiomer, said formulation having a higher content of the (+)-enantiomer compared with the (−)-enantiomer or said formulation having the pure (+)-enantiomer.

43. The method of claim 40, wherein compound A is selected from the group consisting of:
(2RS,3RS)-1-dimethylamino-3-(3-methoxy-phenyl)-2-methyl-pentan-3-ol,
(+)-(2R,3R)-1-dimethylamino-3-(3-methoxy-phenyl)-2-methyl-pentan-3-ol,
(2RS,3RS)-3-(3,4-dichlorophenyl)-1-dimethylamino-2-methyl-pentan-3-ol,
(2RS,3RS)-3-(3-difluoromethyl-phenyl)-1-dimethylamino-2-methyl-pentan-3-ol,
(2RS,3RS)-1-dimethylamino-2-methyl-3-(3-methylsulfanyl-phenyl)-pentan-3-ol,
(3RS)-1-dimethylamino-3-(3-methoxy-phenyl)-4,4-dimethyl-pentan-3-ol,
(2RS,3RS)-3-(3-dimethylamino-1-ethyl-1-hydroxy-2-methyl-propyl)-phenol,
(1RS,2RS)-3-(3-dimethylamino-1-hydroxy-1,2-dimethyl-propyl)-phenol,
(+)-(1R,2R)-3-(3-dimethylamino-1-hydroxy-1,2-dimethyl-propyl)-phenol,
(+)-(1R,2R)-3-(3-dimethylamino-1-hydroxy-1,2-dimethyl-propyl)-phenol,
(−)-(1R,2R)-3-(3-dimethylamino-1-ethyl-2-methyl-propyl)-phenol,
(+)-(1R,2R)-acetic acid 3-dimethylamino-1-ethyl-1-(3-methoxy-phenyl)-2-methyl-propyl ester,
(1RS)-1-(1-dimethylaminomethyl-cyclohexyl)-1-(3-methoxy-phenyl)-propan-1-ol,
(2RS,3RS)-3-(4-chlorophenyl)-1-dimethylamino-2-methyl-pentan-3-ol,
(+)-(2R,3R)-3-(3-dimethylamino-1-ethyl-1-hydroxy-2-methyl-propyl-phenol,
(2RS,3RS)-4-dimethylamino-2-(3-methoxy-phenyl)-3-methyl-butan-2-ol and
(+)-(2R,3R)-4-dimethylamino-2-(3-methoxy-phenyl)-3-methyl-butan-2-ol.

44. The method of claim 43, wherein compound A is in the form of a hydrochloride.

45. The method of claim 29, wherein the compound A in group d) is chosen from compounds corresponding to formula II for which:
X is chosen from OH, F, Cl, OC(O)CH₃ or H;
R¹ is chosen from C₁₋₄-alkyl, CF₃, OH, O—C₁₋₄-alkyl, Cl or F;
R⁹-R¹³, where 3 or 4 of the radicals R⁹ to R¹³ must correspond to H, independently of one another are chosen from H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
or R¹² and R¹¹ form a 3,4-OCH═CH ring
or if R⁹, R¹¹ and R¹³ correspond to H, one of R¹⁰ or R¹² also corresponds to H, while the other is chosen from Cl, F, OH, CF₂H, CF₃, OR¹⁴ or SR¹⁴;
or if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F;
or if R⁹, R¹⁰, R¹² and R¹³ correspond to H, R¹¹ is chosen from CF₃, CF₂H, Cl or F;
or if R¹⁰, R¹¹ and R¹² correspond to H, one of R⁹ or R¹³ also corresponds to H, while the other is chosen from OH, OC₂H₅ or OC₃H₇;
or if R⁹, R¹¹ and R¹³ correspond to H, one of R¹⁰ or R¹² also corresponds to H, while the other is chosen from Cl, F, OH, SH, CF₂H, CF₃, OR¹⁴ or SR¹⁴.

46. A method according to claim 45, wherein compounds corresponding to formula II are present in the form of the diastereomers having the relative configuration IIa 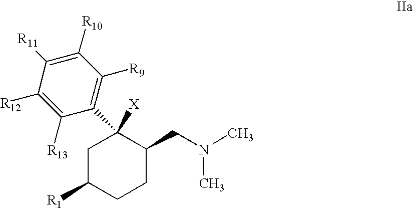
or
compounds corresponding to formula II are present in the form of the (+) enantiomer.

47. A method according to claim 46, wherein compounds corresponding to formula II are present in the form of the diastereomers having the relative configuration Ia in a greater amount than the other diastereomer or the pure diastereomer having the relative configuration IIa is provided or
compounds corresponding to formula II are present in the form of the (+)-enantiomer, said formulation having a higher content of the (+)-enantiomer compared with the (−)-enantiomer or said formulation having the pure (+)-enantiomer.

48. A method according to claim 45, wherein compound A is selected from the group consisting of:
(1RS,3RS,6RS)-6-dimethylaminomethyl-1-(3-methoxy-phenyl)-cyclohexane-1,3-diol,
(+)-(1R,3R,6R)-6-dimethylaminomethyl-1-(3-methoxy-phenyl)-cyclohexane-1,3-diol,
(1RS,3RS,6RS)-6-dimethylaminomethyl-1-(3-hydroxy-phenyl)-cyclohexane-1,3-diol,
(1RS,3SR,6RS)-6-dimethylaminomethyl-1-(3-methoxy-phenyl)-cyclohexane-1,3-diol,
(+)-(1R,2R,5 S)-3-(2-dimethylaminomethyl-1-hydroxy-5-methyl-cyclohexyl)-phenol and
(1RS,2RS,5RS)-3-(2-dimethylaminomethyl-1-hydroxy-5-trifluoromethyl-cyclohexyl)-phenol.

49. A method according to claim 48, wherein compound A is in the form of a hydrochloride.

50. A method according to claim 29, wherein compound A in group e) is chosen from compounds corresponding to formula III for which:
X is chosen from OH, F, Cl, OC(O)CH₃ or H;
R⁹-R¹³, where 3 or 4 of the radicals R⁹ to R¹³ must correspond to H, independently of one another are chosen from H, Cl, F, OH, CF₂H, CF₃ or C₁₋₄-alkyl, saturated and unsubstituted, branched or unbranched; OR¹⁴ or SR¹⁴, where R¹⁴ is chosen from C₁₋₃-alkyl, saturated and unsubstituted, branched or unbranched;
or R¹² and R¹¹ form a 3,4-OCH═CH ring;
or if R⁹, R¹¹ and R¹³ correspond to H, one of R¹⁰ or R¹² also corresponds to H, while the other is chosen from Cl, F, OH, CF₂H, CF₃, OR¹⁴ or SR¹⁴;
or if R⁹ and R¹³ correspond to H and R¹¹ corresponds to OH, OCH₃, Cl or F, preferably Cl, one of R¹⁰ or R¹² also corresponds to H, while the other corresponds to OH, OCH₃, Cl or F;
or if R⁹, R¹⁰, R¹² and R¹³ correspond to H, R¹¹ is chosen from CF₃, CF₂H, Cl or F;
or if R¹⁰, R¹¹ and R¹² correspond to H, one of R⁹ or R¹³ also corresponds to H, while the other is chosen from OH, OC₂H₅ or OC₃H₇;
or if R⁹, R¹¹ and R¹³ correspond to H, one of R¹⁰ or R¹² also corresponds to H, while the other is chosen from Cl, F, OH, SH, CF₂H, CF₃, OR¹⁴ or SR¹⁴.

51. A method according to claim 50, wherein compounds corresponding to formula III are present in the form of their diastereomers having the relative configuration IIIa 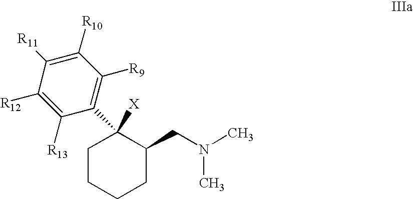
or
compounds corresponding to formula III are present in the form of the (+)-enantiomer.

52. A method according to claim 51, wherein compounds corresponding to formula III are present in the form of the diastereomers having the relative configuration IIIa in a greater amount than the other diastereomer or the pure diastereomer having the relative configuration IIa is provided
or
compounds corresponding to formula III are present in the form of the (+)-enantiomer, said formulation having a higher content of the (+)-enantiomer compared with the (−)-enantiomer or said formulation having the pure (+)-enantiomer.

53. A method according to claim 50, wherein compound A is selected from the group consisting of:
(+)-(1R,2R)-3-(2-dimethylaminomethyl-1-fluoro-cyclohexyl)-phenol,
(+)-(1S,2S)-3-(2-dimethylaminomethyl-cyclohexyl)-phenol and
(−)-(1R,2R)-3-(2-dimethylaminomethyl-cyclohexyl)-phenol.

54. A method according to claim 53, wherein compound A is present in the form of a hydrochloride.

55. A method according to claim 29, wherein compound B is selected from the group consisting of:
fesoterodine, solifenacin (YM905), cizolirtine, resiniferatoxin and venlaxafine.
